(19) United States
(12) Patent Application Publication
(10) Pub. No.: US 2021/0269504 A1
(43) Pub. Date: Sep. 2, 2021

(54) PROTEIN-BASED MICELLES FOR THE DELIVERY OF HYDROPHOBIC ACTIVE COMPOUNDS

(71) Applicant: THE REGENTS OF THE UNIVERSITY OF CALIFORNIA, Oakland, CA (US)

(72) Inventors: Matthew B. FRANCIS, Berkeley, CA (US); Sarah H. KLASS, Oakland, CA (US); Matthew J. SMITH, Gig Harbor, WA (US)

(73) Assignee: THE REGENTS OF THE UNIVERSITY OF CALIFORNIA, Oakland, CA (US)

(21) Appl. No.: 17/258,250

(22) PCT Filed: Jul. 8, 2019
(86) PCT No.: PCT/US2019/040775

§ 371 Date: Jan. 6, 2021

Related U.S. Application Data
(60) Provisional application No. 62/695,474, filed on Jul. 9, 2018.

(51) Int. Cl. C07K 14/705; C07K 14/47; C07K 14/005; C07K 14/395; C07K 14/35; C07K 14/415; A61K 9/107; A01N 25/02; A01N 47/24; C12N 9/90; A61K 31/431; B01J 31/24; B01J 31/22; B01J 31/06; B01J 35/00

(52) U.S. Cl. CPC C07K 14/70567; A61K 38/00; C07K 14/005; C07K 14/395; C07K 14/4747; C07K 14/35; C07K 14/415; A61K 9/1075; A01N 25/02; A01N 47/24; C12N 9/90; C12Y 503/03001; A61K 31/431; B01J 31/2452; B01J 31/2295; B01J 31/062; B01J 35/0013; C07K 14/47

(57) ABSTRACT

An amphiphilic fusion protein has a formula S/I—X—H₁—H₂, wherein S— is a solubilizing moiety, I— is an insolubilizing moiety, —X— is a peptide sequence comprising a proteolytic or chemical cleavage site, —H₁— is a hydrophilic peptide, and —H₂ is a hydrophobic peptide.

### CROSS-REFERENCE TO RELATED APPLICATIONS

The present application claims the benefit of priority to U.S. Application No. 62/695,474, filed on Jul. 9, 2018, the contents of which are incorporated herein in their entirety.

### TECHNICAL FIELD

The present technology relates generally to methods and compositions pertaining to amphiphilic proteins that self-assemble to form stable micelles. Such amphiphilic proteins and corresponding micelles are useful for the delivery of hydrophobic compounds for a variety of applications.

### BACKGROUND

The following description is provided to assist the understanding of the reader. None of the information provided or references cited is admitted to be prior art to the compositions and methods disclosed herein.

Previous studies relating to the recombinant expression of amphiphilic proteins has heavily focused on using naturally self-assembling proteins such as the sunflower protein oleosin, hydrogel formation by leucine zipper proteins, or elastin-like proteins (ELPs), which consist of repeats of the sequence VPGXG (SEQ ID NO: 64). While these constructs can form a variety of 3D structures in vivio and in vitro, they are limited in their ability for functionalization and rely heavily on the use of proteins that are known to naturally self-assemble. Accordingly, there is a need for a more effective and efficient approach to producing amphiphilic proteins.

### SUMMARY

In one aspect, the present disclosure provides an amphiphilic fusion protein having a formula S/I—X—H₁—H₂, wherein S— is a solubilizing moiety, I— is an insolubilizing moiety, —X— is a peptide sequence comprising a proteolytic or chemical cleavage site, —H₁— is a hydrophilic peptide, and —H₂ is a hydrophobic peptide.

In one aspect, the present disclosure provides an amphiphilic fusion protein having a formula S—X—H₁—H₂, wherein S— is a solubilizing moiety, —X— is a peptide sequence comprising a proteolytic cleavage site, —H₁— is a hydrophilic peptide, and —H₂ is a hydrophobic peptide.

In one aspect, the present disclosure provides an amphiphilic fusion protein having a formula I—X—H₁—H₂, wherein I— is an insolubilizing moiety, —X— is a peptide sequence comprising a chemical cleavage site, —H₁— is a hydrophilic peptide, and —H₂ is a hydrophobic peptide.

In some embodiments, the —H₁— comprises an intrinsically disordered peptide (IDP) sequence. In some embodiments, the IDP sequence comprises one or more polypeptide sequences from a human neurofilament protein, a San1 protein, an Hsp-33 protein, an E1A protein, a PhD protein, a Sic1 protein, a WASP protein, a p27 protein, a CREB protein, a PUP protein, or a LEA protein. In some embodiments, the IDP comprises a human neurofilament polypeptide sequence. In some embodiments, the human neurofilament polypeptide sequence comprises the amino acid sequence as set forth in SEQ ID NO: 2. In some embodiments, the IDPs of the present technology comprise the human neurofilament polypeptide sequence as set forth in SEQ ID NO: 69, or fragments thereof. In some embodiments, the IDP comprises repeats of the sequence (SPAEAK)ₙ (SEQ ID NO: 3) or repeats of the sequence (SPAEAR)ₙ (SEQ ID NO: 4), where n is an integer from 2 to 50. In some embodiments, the IDP comprises repeats of the sequence (SPAX₁AX₂). (SEQ ID NO: 53), where X₁ and X₂ are each any charged amino acid and n is an integer from 2 to 50.

In some embodiments, the —H₂ comprises a hydrophobic polypeptide sequence comprising a tyrosine-rich amino acid sequence 5-20 residues in length. In some embodiments, the —H₂ comprises a hydrophobic polypeptide sequence selected from the group consisting of: YGAYAQYVYIYAYWYL (SEQ ID NO: 5), YGAYAQYVYIYAYWYLYAYI (SEQ ID NO: 6), YGAYAQYVYIYAYWYLYAYIAVAL (SEQ ID NO: 54), WEAKLAKALAKALAKHLAKALAKALKACEA (SEQ ID NO: 7), YWCCA(X)ₐ (SEQ ID NO: 8) where a is a number of any hydrophobic residue (X), YWXXV_{b}A_{b} (SEQ ID NO: 9) where b is an integer of 3 or greater and X is any hydrophobic residue, and YWA(X)_{c} (SEQ ID NO: 10) where c is a number of any hydrophobic residue (X).

In some embodiments, the S— comprises one or more of a maltose binding protein (MBP) polypeptide sequence, a small ubiquitin-like modifier (SUMO) polypeptide sequence, a glutathione S-transferase (GST) polypeptide sequence, a SlyD polypeptide sequence, a NusA polypeptide sequence, a thioredoxin polypeptide sequence, a ubiquitin polypeptide sequence, or a T7 gene 10 polypeptide sequence. In some embodiments, the S— further comprises a polyhistidine tag (His-tag). In some embodiments, the S— comprises a MBP polypeptide sequence. In some embodiments, the S— comprises an amino acid sequence set forth in SEQ ID NO: 12.

In some embodiments, the —X— comprises a proteolytic cleavage site selected from a thrombin cleavage site, a tobacco etch virus (TEV) cleavage site, a 3C cleavage site, an enterokinase cleavage site, or a Factor Xa cleavage site. In some embodiments, the proteolytic cleavage site is a thrombin cleavage site comprising the polypeptide sequence LVPR (SEQ ID NO: 13).

In some embodiments, the I— comprises a ketosteroid isomerase polypepide sequence. In some embodiments, the I— comprises an amino acid sequence set forth in SEQ ID NO: 55.

In some embodiments, the —X— comprises a chemical cleavage site selected from a CNBr cleavage site that cleaves at a methionine residue or a 2-nitro-5-thiocyanobenzoic acid cleavage site that cleaves at a cysteine residue.

In some embodiments, the fusion protein further comprises a cell targeting peptide (-T-) between the —X— and the —H₁—, such that the amphiphilic fusion protein has the formula S/I-X-T-H₁—H₂. In some embodiments, the fusion protein further comprises a cell targeting peptide (-T-) between the —X— and the —H₁—, such that the amphiphilic fusion protein has the formula S—X-T-H₁—H₂. In some embodiments, the fusion protein further comprises a cell targeting peptide (-T-) between the —X— and the —H₁—, such that the amphiphilic fusion protein has the formula I-X-T-H₁—H₂. In some embodiments, the -T- is selected from the group consisting of a chitin binding domain (CBD), a cancer cell-targeting peptide, and an antimicrobial peptide. In some embodiments, the -T- is a cancer cell-targeting peptide selected from the group consisting of a peptide targeting human head and neck solid tumors and having the amino acid sequence TSPLNIHNGQKL (SEQ ID NO: 18), a peptide targeting tumor neovasculature and having the amino acid sequence CGKRK (SEQ ID NO: 19), a peptide targeting breast carcinoma and having the amino acid sequence CGNKRTRGC (SEQ ID NO: 20), a peptide targeting prostate vasculature and having the amino acid sequence SMSIARL (SEQ ID NO: 21), a peptide targeting hepatocellular carcinoma cells and having the amino acid sequence FQHPSFI (SEQ ID NO: 22), a peptide targeting integrin receptor and having the amino acid sequence RGD (SEQ ID NO: 23), a peptide targeting tumor neovasculature and having the amino acid sequence NGR (SEQ ID NO: 24), a peptide targeting endothelial VCAM-1 expressing cells and having the amino acid sequence VHSPNKK (SEQ ID NO: 25), a peptide targeting adenocarcinoma cells and having the amino acid sequence RRPYIL (SEQ ID NO: 26), a peptide targeting various carcinoma and having the amino acid sequence EDYELMDLLAYL (SEQ ID NO: 27), a peptide targeting breast carcinoma and having the amino acid sequence LTVSPWY (SEQ ID NO: 28), and a peptide targeting tumor neovasculature and having the amino acid sequence ATWLPPR (SEQ ID NO: 29). In some embodiments, the -T- is an antimicrobial peptide selected from the group consisting of a dermcidin, an apidaecin, a bactenecin, and a pyrrhocoricin. In some embodiments, the dermcidin is a dermcidin variant selected from the group consisting of DCD-1L comprising the amino acid sequence SSLLEKGLDGAKKAVGGLGKLGKDAVEDLESVGKGAVHDVKDVLDSVL (SEQ ID NO: 30), DCD-1 comprising the amino acid sequence SSLLEKGLDGAKKAVGGLGKLGKDAVEDLESVGKGAVHDVKDVLDSV (SEQ ID NO: 31), and SSL25 comprising the amino acid sequence SSLLEKGLDGAKKAVGGLGKLGKDA (SEQ ID NO: 32). In some embodiments, the apidaecin comprises the amino acid sequence GNNRP(V/I)YIPQPRPPHPR(L/I) (SEQ ID NO: 33). In some embodiments, the bactenecin is bactenecin 5 (Bac 5) or bactenecin 7 (Bac 7). In some embodiments, the pyrrhocoricin comprises the amino acid sequence

(SEQ ID NO: 34)
VDKGSYLPRPTPPRPIYNRN.

In some embodiments, the —H₁—H₂ comprises an amino acid sequence selected from the group consisting of SEQ ID NO: 39, SEQ ID NO: 42, SEQ ID NO: 56, and SEQ ID NO: 57.

In one aspect, the present disclosure provides an expression vector comprising a chimeric nucleic acid sequence encoding an amphiphilic fusion protein having a formula S/I—X—H₁—H₂, wherein S— is a solubilizing moiety, I— is an insolubilizing moiety, —X— is a peptide sequence comprising a proteolytic or chemical cleavage site, —H₁— is a hydrophilic peptide, and —H₂ is a hydrophobic peptide. In one aspect, the present disclosure provides an expression vector comprising a chimeric nucleic acid sequence encoding an amphiphilic fusion protein having a formula S—X—H₁—H₂, wherein S— is a solubilizing moiety, —X— is a peptide sequence comprising a proteolytic cleavage site, —H₁— is a hydrophilic peptide, and —H₂ is a hydrophobic peptide. In one aspect, the present disclosure provides an expression vector comprising a chimeric nucleic acid sequence encoding an amphiphilic fusion protein having a formula I—X—H₁—H₂, wherein I— is an insolubilizing moiety, —X— is a peptide sequence comprising a chemical cleavage site, —H₁— is a hydrophilic peptide, and —H₂ is a hydrophobic peptide.

In one aspect, the present disclosure provides a recombinant host cell engineered to express an amphiphilic fusion protein having a formula S/I—X—H₁—H₂, wherein S— is a solubilizing moiety, I— is an insolubilizing moiety, —X— is a peptide sequence comprising a proteolytic or chemical cleavage site, —H₁— is a hydrophilic peptide, and —H₂ is a hydrophobic peptide, wherein the host cell is a eukaryotic, prokaryotic, archaea, mammalian, yeast, bacteria, cyanobacteria, insect, or plant cell. In one aspect, the present disclosure provides a recombinant host cell engineered to express an amphiphilic fusion protein having a formula S—X—H₁—H₂, wherein S— is a solubilizing moiety, —X— is a peptide sequence comprising a proteolytic cleavage site, —H₁— is a hydrophilic peptide, and —H₂ is a hydrophobic peptide, wherein the host cell is a eukaryotic, prokaryotic, archaea, mammalian, yeast, bacteria, cyanobacteria, insect, or plant cell. In one aspect, the present disclosure provides a recombinant host cell engineered to express an amphiphilic fusion protein having a formula I-X—H₁—H₂, wherein I— is an insolubilizing moiety, —X— is a peptide sequence comprising a chemical cleavage site, —H₁— is a hydrophilic peptide, and —H₂ is a hydrophobic peptide, wherein the host cell is a eukaryotic, prokaryotic, archaea, mammalian, yeast, bacteria, cyanobacteria, insect, or plant cell. In some embodiments, the bacteria cell is E. coli.

In one aspect, the present disclosure provides a method of producing an amphiphilic fusion protein that spontaneously self-assembles to form a stable micelle, the method comprising: (a) introducing into a host cell an expression vector comprising a chimeric nucleic acid construct comprising, in the 5′ to 3′ direction, a promoter suitable for directing expression in a host cell operably linked to a nucleic acid sequence encoding an amphiphilic fusion protein having Formula (I): S/I—X—H₁—H₂, wherein S— is a solubilizing moiety, I— is an insolubilizing moiety, —X— is a peptide sequence comprising a proteolytic or chemical cleavage site, —H₁— is a hydrophilic peptide, and —H₂ is a hydrophobic peptide; (b) growing the host cell under conditions that allow for expression of the chimeric nucleic acid to produce the amphiphilic fusion protein; (c) purifying the amphiphilic fusion protein; and (d) contacting the amphiphilic fusion protein with a protease or a reagent to induce chemical cleavage to provide an amphiphilic fusion protein having Formula (II): H₁—H₂.

In some embodiments of the method, the chimeric nucleic acid construct of part (a) encodes an amphiphilic fusion protein further comprising a cell targeting peptide (-T-) between the —X— and the —H₁—, such that the amphiphilic fusion protein has Formula (III): S/I-X-T-H₁—H₂, and such that after part (d) the amphiphilic fusion protein has Formula (IV): T-H₁—H₂.

In some embodiments of the method, the —H₁— comprises an intrinsically disordered peptide (IDP) sequence. In some embodiments, the IDP sequence comprises one or more polypeptide sequences from a human neurofilament protein, a San1 protein, an Hsp-33 protein, an E1A protein, a PhD protein, a Sic1 protein, a WASP protein, a p27 protein, a CREB protein, a PUP protein, or a LEA protein. In some embodiments, the IDP comprises a human neurofilament polypeptide sequence. In some embodiments, the human neurofilament polypeptide sequence comprises the amino acid sequence as set forth in SEQ ID NO: 2. In some embodiments, the IDPs of the present technology comprise the human neurofilament polypeptide sequence as set forth in SEQ ID NO: 69, or fragments thereof. In some embodiments, the IDP comprises repeats of the sequence (SPAEAK)ₙ (SEQ ID NO: 3) or repeats of the sequence (SPAEAR)ₙ (SEQ ID NO: 4), where n is an integer from 2 to 50. In some embodiments, the IDP comprises repeats of the sequence (SPAX₁AX₂)ₙ (SEQ ID NO:53), where X₁ and X₂ are each any charged amino acid and n is an integer from 2 to 50.

In some embodiments of the method, the —H₂ comprises a hydrophobic polypeptide sequence comprising a tyrosine-rich amino acid sequence 5-20 residues in length. In some embodiments, the —H₂ comprises a hydrophobic polypeptide sequence selected from the group consisting of: YGAYAQYVYIYAYWYL (SEQ ID NO: 5), YGAYAQYVYIYAYWYLYAYI (SEQ ID NO: 6), YGAYAQYVYIYAYWYLYAYIAVAL (SEQ ID NO: 54), WEAKLAKALAKALAKHLAKALAKALKACEA (SEQ ID NO: 7), YWCCA(X)ₐ (SEQ ID NO: 8) where a is a number of any hydrophobic residue (X), YWXXV_{b}A_{b} (SEQ ID NO: 9) where b is an integer of 3 or greater and X is any hydrophobic residue, and YWA(X)_{c} (SEQ ID NO: 10) where c is a number of any hydrophobic residue (X).

In some embodiments of the method, the S— comprises one or more of a maltose binding protein (MBP) polypeptide sequence, a small ubiquitin-like modifier (SUMO) polypeptide sequence, a glutathione S-transferase (GST) polypeptide sequence, a SlyD polypeptide sequence, a NusA polypeptide sequence, a thioredoxin polypeptide sequence, a ubiquitin polypeptide sequence, or a T7 gene 10 polypeptide sequence. In some embodiments, the S— further comprises a polyhistidine tag (His-tag). In some embodiments, the S— comprises a MBP polypeptide sequence. In some embodiments, the S— comprises an amino acid sequence set forth in SEQ ID NO: 12.

In some embodiments of the method, the —X— comprises a proteolytic cleavage site selected from a thrombin cleavage site, a tobacco etch virus (TEV) cleavage site, a 3C cleavage site, an enterokinase cleavage site, or a Factor Xa cleavage site. In some embodiments, the proteolytic cleavage site is a thrombin cleavage site comprising the polypeptide sequence LVPR (SEQ ID NO: 13).

In some embodiments, the I— comprises a ketosteroid isomerase polypepide sequence. In some embodiments, the I— comprises an amino acid sequence set forth in SEQ ID NO: 55.

In some embodiments, the —X— comprises a chemical cleavage site selected from a CNBr cleavage site that cleaves at a methionine residue or a 2-nitro-5-thiocyanobenzoic acid cleavage site that cleaves at a cysteine residue.

In some embodiments of the method, the -T- is selected from the group consisting of a chitin binding domain (CBD), a cancer cell-targeting peptide, and an antimicrobial peptide. In some embodiments, the -T- is a cancer cell-targeting peptide selected from the group consisting of a peptide targeting human head and neck solid tumors and having the amino acid sequence TSPLNIHNGQKL (SEQ ID NO: 18), a peptide targeting tumor neovasculature and having the amino acid sequence CGKRK (SEQ ID NO: 19), a peptide targeting breast carcinoma and having the amino acid sequence CGNKRTRGC (SEQ ID NO: 20), a peptide targeting prostate vasculature and having the amino acid sequence SMSIARL (SEQ ID NO: 21), a peptide targeting hepatocellular carcinoma cells and having the amino acid sequence FQHPSFI (SEQ ID NO: 22), a peptide targeting integrin receptor and having the amino acid sequence RGD (SEQ ID NO: 23), a peptide targeting tumor neovasculature and having the amino acid sequence NGR (SEQ ID NO: 24), a peptide targeting endothelial VCAM-1 expressing cells and having the amino acid sequence VHSPNKK (SEQ ID NO: 25), a peptide targeting adenocarcinoma cells and having the amino acid sequence RRPYIL (SEQ ID NO: 26), a peptide targeting various carcinoma and having the amino acid sequence EDYELMDLLAYL (SEQ ID NO: 27), a peptide targeting breast carcinoma and having the amino acid sequence LTVSPWY (SEQ ID NO: 28), and a peptide targeting tumor neovasculature and having the amino acid sequence ATWLPPR (SEQ ID NO: 29). In some embodiments, the -T- is an antimicrobial peptide selected from the group consisting of a dermcidin, an apidaecin, a bactenecin, and a pyrrhocoricin. In some embodiments, the dermcidin is a dermcidin variant selected from the group consisting of DCD-1L comprising the amino acid sequence SSLLEKGLDGAKKAVGGLGKLGKDAVEDLESVGKGAVHDVKDVLDSVL (SEQ ID NO: 30), DCD-1 comprising the amino acid sequence SSLLEKGLDGAKKAVGGLGKLGKDAVEDLESVGKGAVHDVKDVLDSV (SEQ ID NO: 31), and SSL25 comprising the amino acid sequence SSLLEKGLDGAKKAVGGLGKLGKDA (SEQ ID NO: 32). In some embodiments, the apidaecin comprises the amino acid sequence GNNRP(V/I)YIPQPRPPHPR(L/I) (SEQ ID NO: 33). In some embodiments, the bactenecin is bactenecin 5 (Bac 5) or bactenecin 7 (Bac 7). In some embodiments, the pyrrhocoricin comprises the amino acid sequence

(SEQ ID NO: 34)
VDKGSYLPRPTPPRPIYNRN.

In some embodiments of the method, the —H₁—H₂ comprises an amino acid sequence selected from the group consisting of SEQ ID NO: 39, SEQ ID NO: 42, SEQ ID NO: 56, and SEQ ID NO: 57.

In one aspect, the present disclosure provides a micelle comprising an amphiphilic fusion protein comprising: (i) a hydrophilic peptide (H₁); and (ii) a hydrophobic peptide (H₂).

In some embodiments, the H₁ comprises an intrinsically disordered peptide (IDP) sequence. In some embodiments, the IDP sequence comprises one or more polypeptide sequences from a human neurofilament protein, a San1 protein, an Hsp-33 protein, an E1A protein, a PhD protein, a Sic1 protein, a WASP protein, a p27 protein, a CREB protein, a PUP protein, or a LEA protein. In some embodiments, the IDP comprises a human neurofilament polypeptide sequence. In some embodiments, the human neurofilament polypeptide sequence comprises the amino acid sequence as set forth in SEQ ID NO: 2. In some embodiments, the IDPs of the present technology comprise the human neurofilament polypeptide sequence as set forth in SEQ ID NO: 69, or fragments thereof. In some embodiments, the IDP comprises repeats of the sequence (SPAEAK)ₙ (SEQ ID NO: 3) or repeats of the sequence (SPAEAR)ₙ (SEQ ID NO: 4), where n is an integer from 2 to 50. In some embodiments, the IDP comprises repeats of the sequence (SPAX₁AX₂)ₙ (SEQ ID NO: 53), where X₁ and X₂ are each any charged amino acid and n is an integer from 2 to 50.

In some embodiments, the H₂ comprises a hydrophobic polypeptide sequence comprising a tyrosine-rich amino acid sequence 5-20 residues in length. In some embodiments, the H₂ comprises a hydrophobic polypeptide sequence selected from the group consisting of: YGAYAQYVYIYAYWYL (SEQ ID NO: 5), YGAYAQYVYIYAYWYLYAYI (SEQ ID NO: 6), WEAKLAKALAKALAKHLAKALAKALKACEA (SEQ ID NO: 7), YWCCA(X)ₐ (SEQ ID NO: 8) where a is a number of any hydrophobic residue (X), YWXXV_{b}A_{b} (SEQ ID NO: 9) where b is an integer of 3 or greater and X is any hydrophobic residue, and YWA(X)_{c} (SEQ ID NO: 10) where c is a number of any hydrophobic residue (X).

In some embodiments, the amphiphilic fusion protein further comprises a cell targeting peptide (T) covalently linked to the N-terminus of the H₁. In some embodiments, the T is selected from the group consisting of a chitin binding domain (CBD), a cancer cell-targeting peptide, and an antimicrobial peptide. In some embodiments, the cancer cell-targeting peptide is selected from the group consisting of a peptide targeting human head and neck solid tumors and having the amino acid sequence TSPLNIHNGQKL (SEQ ID NO: 18), a peptide targeting tumor neovasculature and having the amino acid sequence CGKRK (SEQ ID NO: 19), a peptide targeting breast carcinoma and having the amino acid sequence CGNKRTRGC (SEQ ID NO: 20), a peptide targeting prostate vasculature and having the amino acid sequence SMSIARL (SEQ ID NO: 21), a peptide targeting hepatocellular carcinoma cells and having the amino acid sequence FQHPSFI (SEQ ID NO: 22), a peptide targeting integrin receptor and having the amino acid sequence RGD (SEQ ID NO: 23), a peptide targeting tumor neovasculature and having the amino acid sequence NGR (SEQ ID NO: 24), a peptide targeting endothelial VCAM-1 expressing cells and having the amino acid sequence VHSPNKK (SEQ ID NO: 25), a peptide targeting adenocarcinoma cells and having the amino acid sequence RRPYIL (SEQ ID NO: 26), a peptide targeting various carcinoma and having the amino acid sequence EDYELMDLLAYL (SEQ ID NO: 27), a peptide targeting breast carcinoma and having the amino acid sequence LTVSPWY (SEQ ID NO: 28), and a peptide targeting tumor neovasculature and having the amino acid sequence ATWLPPR (SEQ ID NO: 29). In some embodiments, the T is an antimicrobial peptide selected from the group consisting of a dermcidin, an apidaecin, a bactenecin, and a pyrrhocoricin. In some embodiments, the dermcidin is a dermcidin variant selected from the group consisting of DCD-1L comprising the amino acid sequence SSLLEKGLDGAKKAVGGLGKLGKDAVEDLESVGKGAVHDVKDVLDSVL (SEQ ID NO: 30), DCD-1 comprising the amino acid sequence SSLLEKGLDGAKKAVGGLGKLGKDAVEDLESVGKGAVHDVKDVLDSV (SEQ ID NO: 31), and SSL25 comprising the amino acid sequence SSLLEKGLDGAKKAVGGLGKLGKDA (SEQ ID NO: 32). In some embodiments, the apidaecin comprises the amino acid sequence GNNRP(V/I)YIPQPRPPHPR(L/I) (SEQ ID NO: 33). In some embodiments, the bactenecin is bactenecin 5 (Bac 5) or bactenecin 7 (Bac 7). In some embodiments, the pyrrhocoricin comprises the amino acid sequence

(SEQ ID NO: 34)
VDKGSYLPRPTPPRPIYNRN.

In some embodiments, the critical micelle concentration (CMC) of the amphiphilic fusion protein in water is from about 10 μM to about 20 μM at a physiological pH of about 7.4.

In some embodiments, the micelle has a diameter from about 20 nm to about 40 nm. In some embodiments, the micelle has a diameter of about 27 nm.

In some embodiments, the micelle is stable at a pH from about 2.0 to about 10.0.

In some embodiments, the micelle is stable at a temperature from about 25° C. to about 70° C.

In some embodiments, the micelle further comprises a fluorescent dye. In some embodiments, the fluorescent dye is covalently attached to the hydrophilic peptide (H₁). In some embodiments, the fluorescent dye is covalently attached to the hydrophobic peptide (H₂). In some embodiments, the fluorescent dye is fluorescein or rhodamine.

In some embodiments, the micelle has a core-shell structure. In some embodiments, the micelle has a shell diameter from about 40 nm to about 75 nm. In some embodiments, the micelle has a core diameter from about 25 nm to about 45 nm. In some embodiments, the micelle has a shell thickness from about 5 nm to about 20 nm.

In some embodiments, the micelle further comprises a hydrophobic cargo. In some embodiments, the hydrophobic cargo is a drug, a fungicide, a protein, a nucleic acid, a hormone, a receptor, a diagnostic agent, an imaging agent, a metal complex, a silicone oil, a triglyceride, or a combination thereof.

In some embodiments, the amphiphilic fusion protein comprising H₁ and H₂ comprises an amino acid sequence selected from the group consisting of SEQ ID NO: 39, SEQ ID NO: 42, SEQ ID NO: 56, and SEQ ID NO: 57.

In one aspect, the preset disclosure provides a pharmaceutical composition comprising the micelle of the present technology and a hydrophobic cargo, wherein the hydrophobic cargo is a therapeutically active agent.

In one aspect, the preset disclosure provides a method for treating a disease or disorder in a subject in need thereof comprising administering the pharmaceutical composition to the subject.

In one aspect, the preset disclosure provides a composition suitable for use in drug delivery, cosmetics, paints and coatings, crop protection, nanoparticle synthesis and catalysis, home and personal care, and cleaning, comprising the micelle of the present technology.

### BRIEF DESCRIPTION OF THE DRAWINGS

FIG. 1 shows hydrophobicity plots of the three constructs described herein. Values in red correspond to regions of the protein sequence assigned a negative or hydrophilic value whereas blue corresponds to a hydrophobic region. The sequences of the 3 proteins are identical until the C-terminal region, at which each has been modified to contain appendages with an increasing hydrophobic portion.

FIGS. 2A and 2B. FIG. 2A shows a 4-12% Bis-Tris SDS PAGE Gel analysis of NiNTA purified 2Yx2A-MBP proteins under different IPTG induction conditions and either 20 or 6 hour time points. All cultures were expressed at 16° C. Lanes, right to Left: MW Ladder, 20 h 0.5 mM IPTG, 20 h 0.2 mM IPTG, 20 h 0.1 mM IPTG, 6 h 0.5 mM IPTG, 6 h 0.2 mM IPTG, 6 h 0.1 mM IPTG. FIG. 2B shows a LC-MS (ESI-TOF) analysis of NiNTA purified construct with most stringent expression conditions: 6 h 0.1 mM IPTG. Reducing the time of expression and amount of IPTG reduces protein yield but enhances protein purity. Expected molecular weight (with N-terminal Met cleavage): 63604.62, observed: 63605.

FIGS. 3A-3D. FIG. 3A shows a 4-12% Bis-Tris SDS PAGE Gel analysis of ion exchange purified 2Yx2A (75% pure by gel densitometry analyzed in ImageJ). FIG. 3B shows a 4-12% Bis-Tris SDS PAGE Gel analysis of Biotage HPLC purified 2Yx2A shows a single band corresponding to 2Yx2A monomer while also a large band that does not travel down the gel corresponding to the assembled protein that was not disassembled on the PAGE gel (>95% pure by densitometry analyzed in ImageJ). In both gels 2Yx2A complex runs at a higher apparent molecular weight, a phenomenon also observed with the IDP construct, which is likely due to its disordered nature. FIG. 3C shows a purification of 2Yx2A from MBP on poroshell column. 2Yx2A elutes at 8.2 minutes while MBP elutes at 10 min. A small amount of 2Yx2A also elutes around 9 minutes likely due to interactions with MBP. Only pure fractions are collected, for higher throughput purification, a C18 Biotage SNAP Bio 300A is used on a Biotage HPLC setup. FIG. 3D shows an LC-MS (ESI-TOF) analysis of ion exchange purified and HPLC purified 2Yx2A. The expected molecular weight of monomer: 18290.69. For the ion exchange purified protein 70% exists as a monomer (18291 Da), 10% as a dimer (36580 Da), and 19% impurity by MBP (45332 Da). For the Biotage HPLC purified protein, 76% exists as a monomer (cysteine residue capped by excess B-mercaptoethanol in the buffer at +76: 18367 Da), 23% as a dimer (36580 Da), and 0% impurity by MBP (45332 Da).

FIGS. 4A-4C. FIG. 4A is a photograph showing that after cell lysis, sonication, and filtration, the 2Yx3A-MBP crude protein mixture is very soapy. FIG. 4B is a photograph showing that after NiNTA purification of 2Yx3A-MBP construct, the protein mixture is still very soapy. FIG. 4C top graph: LC-MS (ESI-TOF) analysis of NiNTA purified 2Yx3A-MBP shows an impure mixture containing truncations of the 2Yx3A-MBP protein where the desired construct is obtained at 88% purity. Expected molecular weight 2Yx3A-MBP: 64115.21 or 64191.21 (cysteine residue capped by excess B-mercaptoethanol in buffer+76). Observed molecular weight 64193. FIG. 4C middle graph: LC-MS (ESI-TOF) analysis of 2Yx3A+MBP directly after cleavage by thrombin. Molecular weights corresponding to MBP: 45332 as well as 2Yx3A monomer: 18802, dimer: 37602, and trimer: 56401 are observed indicating that this construct has a high propensity to assemble even in the presence of solubilizing MBP, staying in contact even during LC-MS TOF analysis. FIG. 4C bottom graph: Ion exchange purified 2Yx3A. Due to the ability of this construct to assemble even in the presence of MBP, purification of the construct from MBP becomes a challenge. Expected molecular weight of monomer: 18801.28 or 18877.26 (+B-mercaptoethanol). For the ion exchange purified protein 19% exists as monomer: 18802+18878, 18% as dimer: 37601, and 63% impurity by MBP: 45333.

FIGS. 5A-5C. Design of an amphiphilic protein construct. FIG. 5A: An intrinsically disordered protein (IDP) segment is fused to a hydrophobic sequence. Following cleavage of the MBP protein, the amphiphilic portion self-assembles. FIG. 5B: Hydrophobicity plots of the designed sequences are shown, following cleavage of the MBP regions. The values are from the Kyte-Doolittle hydrophobicity scale with a window size of 9. Values greater than 0 indicate a hydrophobic region while those less than zero are hydrophilic. The plots were generated using the Expasy ProtScale tool (web.expasy.org/protscale). FIG. 5C: The specific hydrophobic sequence regions are shown for the constructs used in this report. The c-terminal residues of IDP (YWCA) (SEQ ID NO: 65) are shown and the hydrophobic extensions are underlined (SEQ ID NOs: 65, 66, 67, and 68 in order of appearance).

FIG. 6 is a chart showing DLS measurements of IDP (2 μM in Phosphate Buffer pH 5.3) and 2Yx2A construct (40 μM in 100 mM Phosphate buffer pH 5.3). Average diameters by % number IDP: 11.25±0.80 nm and 2Yx2A: 27.02±1.06 nm.

FIGS. 7A and 7B are charts showing the pH stability of the 2Yx2A construct of the present technology. FIG. 7A is a chart showing DLS measurements of lyophilized 2Yx2A protein resuspended to a concentration of 40 μM in phosphate buffer at pH values ranging from 3.7-9.7 and buffer concentrations ranging from 0-200 mM. Over all pH and buffer concentrations (186 measurements), the average diameter is 26.17+/−4.28 nm. FIG. 7B is a chart summarizing DLS measurements from FIG. 7A. No obvious size dependence on pH is observed. It appears as though at some pHs, such as pH 9.7, increasing phosphate buffer leads to an increase in size while others such as pH 7.2 and 7.9 appear to undergo a collapse at higher potassium phosphate conditions. Interestingly at low pH, no dependence is observed for the addition of potassium phosphate buffer.

FIG. 8 is a chart showing the dependence of 2Yx2A micelle size on the concentration in 1×PBS pH 7.4 and 100 mM PB pH 5.3. As the concentration of protein is decreased, an increase in average size by DLS is observed. Additionally, the standard deviation with each measurement set increases with decreasing concentration indicating a more polydisperse sample. In 1×PBS above a protein concentration of 10 μM, a low standard deviation is observed with diameters that are in accordance with what is seen at higher concentrations (average of 10 and 30 μM samples 28.86±3.37 nm). When the data is fit to a logarithmic graph, an EC₅₀ value of 3.5 μM can be calculated with an R² of 0.92. When 2Yx2A in place in 100 mM PB pH 5.7, the concentration at which it forms micelles with a low polydispersity and average diameter close to 27 nm is considerably lower than when in 1×PBS which has a pH of 7.4. When the data is fit to a logarithmic graph, an EC50 value of 0.0035 μM can be calculated with an R² of 0.84. These trends closely reflect that of the CMC determined by the pyrene fluorescence assay.

FIGS. 9A and 9B are charts showing the effects of temperature on the diameter of 2Yx2A micelles. FIG. 9A is a chart showing DLS measurements of 40 μM 2Yx2A in 100 mM PB pH 5.3 as the temperature is increased. As the temperature increases, the average diameter of the 2Yx2A micelles decreases. Additionally, the error bars become smaller as temperature increases. Diameter at 25° C.: 27.02±1.06 nm diameter at 70° C.: 16.5±0.49 nm. FIG. 9B is a chart showing that after the sample was heated to 70° C. it was let cool back down to room temperature and analyzed again 1 week later at 25° C. at which point it returned to the larger diameter that was observed before it was heated. The average diameter before heating (blue trace): 27.02±1.06 nm average diameter after heating (red trace): 33.99±1.50 nm.

FIG. 10 is a chart showing size exclusion chromatography LS9 traces of virus-like particle MS2 (known diameter 27 nm), IDP, and 2Yx2A micelles. The major peak for the 2Yx2A micelles overlaps that of MS2, further supporting the diameter reported from DLS measurements of 27.73 nm. IDP which shows a diameter of 11.25 nm on the DLS also elutes late indicating a smaller size. Traces have been normalized to maximum peak height; however, it should be noted that the LS90 trace for IDP had a very low intensity reflecting what would be expected of a monomeric protein.

FIGS. 11A and 11B are charts showing the fluorescence emission spectrum of 2Yx2A incubated with pyrene at different concentrations of pyrene. FIG. 11A shows the fluorescence emission spectra of 2Yx2A incubated with 2 μM pyrene in 100 mM PB pH 5.7. As protein concentration is decreased from 100 μM to 0 μM, a decrease in the intensity of the third vibronic band of pyrene is observed indicating that with decreasing protein concentration, pyrene is in an increasingly hydrophilic environment. FIG. 11B shows the first vibronic band of pyrene sits at approximately 372 nm but undergoes a red shift when in hydrophobic environments. The third vibronic band emerges at 383 nm. Additionally, the fifth vibronic band of pyrene also undergoes a red shift when in the presence of a hydrophobic environment which occurs around 394 nm.

FIG. 12 is a chart showing when the ratio of the first to third vibronic bands of pyrene emission is plotted against 2Yx2A and IDP protein concentrations, a Boltzmann relationship is observed for 2Yx2A, where the EC₅₀ is calculated to be 27.6 μM, while encapsulation of pyrene and I3 band formation is observed down to 10 μM. This indicates that the CMC of the 2Yx2A micelles is in the low μM range consistent with the DLS results of FIG. 8 where an increase in size and polydispersity are observed below 10 μM when in 1×PBS. Alternatively, when in 100 mM PB pH 5.7, a more stable pH as indicated by our DLS analysis, the EC₅₀ value drops to 12.96 μM. Additionally, when the same analysis is applied to IDP, no dependence on concentration is observed, with the I1/I3 ratio remaining constant between 0-100 μM. Due to the low CMC of these particles (low μM range), the pyrene assay is only able to provide an upper bound for the detection of the CMC. This data should be examined in conjugation with the DLS data (FIG. 8) as well as experimental evidence of micelle formation at 0.4 μM by cryo TEM (FIG. 15).

FIG. 13 shows 2Yx2A proteins labeled at 4% with either Rhodamine Red dye (top) or Fluorescein dye (bottom).

FIGS. 14A and 14B are charts showing the FRET analysis of the 2Yx2A. FIG. 14A is a FRET analysis of 2Yx2A when excited with 490 nm light the emission of fluorescein is observed at 515 nm whereas the emission of rhodamine is observed at 580 nm. Due to a slight amount of fluorescence observed with only the 2Yx2A-RhoRed complex at 580 nm when excited with 490 nm light, time point zero is taken as the second trace 1:1 FITC-2Yx2A:RhoRED-2Yx2A for the kinetic measurements. FIG. 14B is a chart demonstrating that the FRET ratio, defined at I580/(I580+I515) can be plotted against time and fit to a logarithmic equation. By 75 minutes, 50% mixing of the micelles is achieved in 1×PBS, indicating that the micelles of the present technology are dynamic in nature.

FIG. 15 are photographs showing Cryo TEM of 4 μM 2Yx2A micelles in 100 mM PB pH 5.3. Average micelle size 50.46±12.14 nm. Micelle size is comparable to that of DLS taken prior to analysis 48.43±10.62 nm. A core-shell structure can be observed in some micelles, possibly outlining the transition between the packed micelle interior and the intrinsically disordered hydrophilic shell.

FIG. 16 is a chart showing core-shell diameters of 10 micelles. With increasing core size, there is an increase in shell size. The thickness, defined as the distance between an individual micelles core and shell, for these micelles was on average 12.23±3.95 nm, which is close to the expected length of the intrinsically disordered hydrophilic region of the construct. IDP by DLS: 11.25±0.80 nm.

FIGS. 17A and 17B are charts showing the Rg and P(r) distribution of the 2Yx2A. FIG. 17A is a chart showing SAXS scattering curve of 68 and 34 μM 2Yx2A in 100 mM PB pH 5.7 and 32 μM 2Yx2A in 1×PBS. The fit of the curve is used to determine the real space Rg and the P(r) distribution. FIG. 17B is a chart showing results of the P(r) distribution fit. All three curves appear very similar resulting in real space Rg values that are all approximately 10 nm. The Rg/Rh can give insight to the structural properties of the specific sample, for example, a value of 0.775 indicates a hard sphere whereas larger numbers indicate nonspherical and elongated samples. The Rh obtained from DLS measurements is 13.08 nm resulting in an Rg/Rh ratio of 0.76, consistent with a packed spherical micelle. Additionally, the average radius can be determined for the three samples, where they all show maximum probability between 10 and 15 nm and going to zero probability (dmax) around 320 nm.

FIGS. 18A and 18B show the corresponding HPLC analysis used to determine the amount of pyraclostrostrobin encapsulated in 2Yx2A protein. FIG. 18A shows the calibration curve developed using known pyrene concentrations in acetonitrile. FIG. 18B shows the HPLC analysis of a known amount of protein-pyraclostrobin solution injected onto the HPLC. Based on the area of the pyrene peak and volume injected the number of moles and thus the concentration of pyraclostrobin can be determined. Using this method where pyraclostrobin was directly added to 2Yx2A, 7.37 μM pyraclostrobin is encapsulated in 11 μM of protein.

FIG. 19 is a chart showing the comparison of the number of moles of pyraclostrobin in a sample with and without the 2Yx2A protein present. For the sample with 2Yx2A, the lyophilized protein had been re-suspended with pyraclostrobin in 10 μL THF then diluted with 40 μL of 100 mM PB pH 5.7 to a final concentration of 3 μM. The number of moles of pyraclostrobin and 2Yx2A was determined from HPLC calibration curves. Pyraclostrobin resuspending with 2Yx2A resulted in an average of 0.63 nmol pyraclostrobin injected on the HPLC while pyraclostrobin resuspending in water resulted in 0.03 nmol pyraclostrobin injected on the HPLC. For the sample containing 2Yx2A, the average mole ratio of Pyraclostrobin: 2Yx2A protein monomers was determined to be 15.2±8:1.

FIGS. 20A and 20B are photographs showing unstained TEM images of 2Yx2A micelles loaded with Pd(dppf)Cl₂. Over 4000 particles were analyzed using ImageJ giving an average diameter of 14.9±8 nm.

FIG. 21 shows the SDS PAGE of KSI-IDP-2Yx2A protein purified by centrifugation. The KSI-IDP-2Yx2A protein resides in a relatively pure form in the insoluble fraction after cell lysis. Using only centrifugation as a means of purification, the gel indicates that the KSI-IDP-2Yx2A protein is the predominant species in the insoluble fraction.

FIG. 22 shows the LCMS analysis of KSI-IDP-2Yx2A protein purified by centrifugation. The KSI-IDP-2Yx2A protein resides in a relatively pure form in the insoluble fraction after cell lysis. Using only centrifugation as a means of purification, LCMS analysis indicates that the KSI-IDP-2Yx2A protein is the predominant species in the insoluble fraction, expected molecular weight 32328.60 Da, observed molecular weight: 32328 Da.

FIG. 23 shows the LCMS analysis of CNBr cleaved KSI-IDP-2Yx2A. After cleavage by CNBr overnight, none of the original mass corresponding to KSI-IDP-2Yx2A (32328 Da) is observed. Masses corresponding to expected molecular weight (17631.04 Da) for IDP-2Yx2A (17631 Da) and its dimer (35259 Da) are observed.

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

### I. Definitions

The following terms are used herein, the definitions of which are provided for guidance.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the present technology. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The term “administered” or “administration” when used in the context of therapeutic and diagnostic uses, refers to and includes the introduction of a selected amount of the micelles described herein into an in vivo or in vitro environment for the purpose of, for example, delivering a therapeutic agent to a targeted site. Administration can be carried out by any suitable route, including but not limited to, intravenously, intramuscularly, intraperitoneally, subcutaneously, and other suitable routes as described herein. Administration includes self-administration and the administration by another.

As used herein, “amphiphilic fusion protein” refers to a protein created by the joining of translational sequences from two or more different genes to create one contiguous hybrid or chimeric protein molecule comprising a hydrophobic domain in translational fusion with a hydrophilic domain. The amphiphilic fusion proteins of the present technology may also comprise a solubilizing domain and a proteolytic cleavage site in translational fusion with the hydrophobic domain. In some embodiments, the amphiphilic fusion proteins of the present technology further comprise a cell targeting peptide in translational fusion with the hydrophobic domain. In some embodiments, “amphiphilic fusion protein” refers to micelles comprising the amphiphilic fusion proteins.

The term “cell targeting peptide” refers to a peptide that is conventionally used in the art to recognize and bind specific cells and tissues. In some embodiments, the amphiphilic fusion peptides of the present technology, which form stable micelles, may be conjugated to one or more cell targeting peptides to achieve targeted delivery of an agent or hydrophobic cargo to specific cells and tissues.

A “chimeric nucleic acid” comprises a coding sequence or fragment thereof linked to a nucleotide sequence that is different from the nucleotide sequence with which it is associated in cells in which the coding sequence occurs naturally.

As used herein, the terms “effective amount” or “therapeutically effective amount” or “pharmaceutically effective amount” refer to a quantity sufficient to achieve a desired therapeutic and/or prophylactic effect, e.g., an amount which results in the prevention of, disease, condition and/or symptom(s) thereof. In the context of therapeutic or prophylactic applications, the amount of a composition administered to the subject will depend on the type and severity of the disease and on the characteristics of the individual, such as general health, age, sex, body weight and tolerance to the composition drugs. It will also depend on the degree, severity and type of disease or condition. The skilled artisan will be able to determine appropriate dosages depending on these and other factors. In some embodiments, multiple doses are administered. Additionally or alternatively, in some embodiments, multiple therapeutic compositions or compounds are administered.

“Heterologous nucleic acid” refers to a nucleic acid, DNA, or RNA, which has been introduced into a cell, and which is not a copy of a sequence naturally found in the cell into which it is introduced. Such heterologous nucleic acid may comprise segments that are a copy of a sequence that is naturally found in the cell into which it has been introduced, or fragments thereof.

As used herein, a recombinant or engineered “host cell” refers to a cell e.g., eukaryotic, prokaryotic, yeast, bacteria, such as Escherichia coli, cyanobacteria, insect, plant, archaea, cell-free, or mammalian cell, that has been modified such that it produces fusion proteins of the present technology. In some embodiments, the host cells are in vitro, cultured cells. In some embodiments, the recombinant host cell comprises one or more polynucleotides, each polynucleotide encoding an amphiphilic fusion protein of the present technology or portions thereof.

As used herein, “hydrophobic cargo” refers to any hydrophobic compound or agent that is suitable for delivery by the micelles described herein. Examples of suitable hydrophobic cargo include but are not limited to a drug, a fungicide, a protein, a nucleic acid, a hormone, a receptor, a diagnostic agent, an imaging agent, a metal complex, a silicone oil, a triglyceride, or a combination thereof. Hydrophobic cargo may include hydrophobic agents that are biologically and/or pharmaceutically active.

As used herein, “insolubilizing moiety” refers to a moiety, such as a peptide, that enhances the insolubility of the amphiphilic proteins described herein and in some instances, prevents the amphiphilic protein from undergoing self-assembly to form a micelle. In some embodiments, the insolubilizing moiety comprises a ketosteroid isomerase polypeptide sequence. In some embodiments, the insolubilizing moiety comprises an amino acid sequence as set for in SEQ ID NO: 55. In some embodiments, the insolubilizing moiety is a peptide that further contains a chemical cleavage site and is cleavable. In some embodiments, the chemical cleavage site selected from a CNBr (cyanogen bromide) cleavage site that cleaves at a methionine residue or a 2-nitro-5-thiocyanobenzoic acid cleavage site that cleaves at a cysteine residue.

As used herein, “intrinsically disordered proteins (IDPs), also known as intrinsically unstructured proteins (IUPs), are characterized by the lack of a stable tertiary structure under physiological conditions. In some embodiments, the IDP comprises a polypeptide sequence selected from a human neurofilament protein, San1 protein, Hsp-33 protein, E1A protein, PhD protein, Sic1 protein, WASP protein, p27 protein, CREB protein, PUP protein, LEA protein, or portions or fragments thereof containing intrinsically disordered regions. In some embodiments, the IDPs of the present technology comprise the human neurofilament polypeptide sequence as set forth in SEQ ID NO: 2, or fragments thereof. In some embodiments, the IDPs of the present technology comprise the human neurofilament polypeptide sequence as set forth in SEQ ID NO: 69, or fragments thereof. In some embodiments, the IDPs of the present technology comprise repeats of the sequence (SPAEAK)ₙ (SEQ ID NO: 3), where n is an integer from 2 to 100, or any range in between, such as 2 to 50, or 2 to 25. In some embodiments, n is 25. In some embodiments, the IDPs of the present technology comprise repeats of the sequence (SPAEAR)_{d} (SEQ ID NO: 4), where d is an integer from 2 to 100, or any range in between, such as 2 to 50, or 2 to 25. In some embodiments, d is 25. In some embodiments, the IDP comprises repeats of the sequence (SPAX₁AX₂)ₙ (SEQ ID NO: 53), where X₁ and X₂ are each any charged amino acid and n is an integer from 2 to 100, or any range in between, such as 2 to 50, or 2 to 25. In some embodiments, n is 25.

As used herein, the term “purify,” “purified,” or “purification” means the removal or isolation of a molecule from its environment by, for example, isolation or separation.

As used herein, the term “recombinant polypeptide” refers to a polypeptide that is produced by recombinant DNA techniques, wherein generally DNA encoding the expressed protein or RNA is inserted into a suitable expression vector and that is in turn used to transform a host cell to produce the polypeptide or RNA.

As used herein, “solubilizing moiety” refers to a moiety, such as a peptide, that enhances the solubility of the amphiphilic proteins described herein and in some instances, prevents the amphiphilic protein from undergoing self-assembly to form a micelle. In some embodiments, the solubilizing moiety comprises one or more of a maltose binding protein (MBP) polypeptide sequence, a small ubiquitin-like modifier (SUMO) polypeptide sequence, a glutathione S-transferase (GST) polypeptide sequence, a SlyD polypeptide sequence, a NusA polypeptide sequence, a thioredoxin polypeptide sequence, a ubiquitin polypeptide sequence, or a T7 gene 10 polypeptide sequence. In some embodiments, the solubilizing moiety further comprises a polyhistidine tag (His-tag), such as a 6×His tag. In some embodiments, the solubilizing moiety comprises an amino acid sequence as set for thein SEQ ID NO: 12. In some embodiments, the solubilizing moiety is a peptide that further contains a proteolytic cleavage site and is cleavable. In some embodiments, the proteolytic cleavage site is selected from a thrombin cleavage site (e.g., LVPR; SEQ ID NO: 13), a tobacco etch virus cleavage site (e.g., ENLYFQ; SEQ ID NO: 14), a 3C cleavage site (e.g., LEVLFQ; SEQ ID NO: 15), an enterokinase cleavage site (e.g., DDDDK; SEQ ID NO: 16), or a Factor Xa cleavage site (e.g., IEGR; SEQ ID NO: 17).

As used herein, the term “subject” refers to any animal (e.g., a mammal), including, but not limited to, humans, non-human primates, rodents, and the like (e.g., which is to be the recipient of a particular treatment, or from whom cells are harvested).

The term “therapeutic active agent,” and similar terms referring to a therapeutic or medicinal function mean that the referenced small molecule, macromolecule, protein, nucleic acid, growth factor, hormone, drug, other substance, cell, metal complex, a silicone oil, a triglyceride, or combination thereof can beneficially affect the initiation, course, and/or one or more symptoms of a disease or condition in a subject, and may be used in conjunction with the micelles described herein in the manufacture of medicaments for treating a disease or other condition. Suitable therapeutic agents for encapsulation in the micelles described herein include hydrophobic therapeutic agents.

The terms “treating”, “treat” and “treatment” can include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, the term “vector” or “expression vector” refers to a nucleic acid molecule capable of directing the expression of genes to which they are operatively linked. In general, expression vectors of utility in recombinant DNA techniques are often in the form of “plasmids,” which refer generally to circular double stranded DNA loops that, in their vector form, are not bound to the chromosome. The terms “plasmid” and “vector” are used interchangeably herein. The expression vectors described herein include a polynucleotide sequence described herein in a form suitable for expression of the polynucleotide sequence in a host cell. It will be appreciated by those skilled in the art that the design of the expression vector can depend on such factors as the choice of the host cell to be transformed, the level of expression of polypeptide desired, etc. The expression vectors described herein can be introduced into host cells to produce polypeptides, including fusion polypeptides such as amphiphilic fusion proteins, encoded by the polynucleotide sequences as described herein.

As used herein, “IDP₁-2Yx2A” refers to SEQ ID NO: 39 or micelles comprising SEQ ID NO: 39, depending on the context in which it is used. In some embodiments, “IDP₂-2Yx2A” refers to SEQ ID NO: 56 or micelles comprising SEQ ID NO: 56, depending on the context in which it is used. As used herein, “IDP-2Yx3A” refers to SEQ ID NO: 42 or micelles comprising SEQ ID NO: 42, depending on the context in which it is used. As used herein, “IDP-2Yx4A” refers to SEQ ID NO: 57 or micelles comprising SEQ ID NO: 57.

### II. Amphiphilic Proteins and Corresponding Protein-Based Micelles

Recent efforts relating to the production of amphiphilic proteins that self-assemble have been focused on using naturally self-assembling proteins, which requires the use of proteins that are known to naturally self-assemble and limits the potential for further functionalization. Other approaches have focused on the use of polymers, small molecules, and peptides. These approaches are relatively cost-inefficient and laborious.

To address these shortcomings, the present technology relates to a series of biodegradable amphiphilic fusion proteins comprising an intrinsically disordered protein (IDP) segment that are produced through a biological mechanism. Accordingly, provided herein in one aspect are recombinant amphiphilic proteins that self-assemble to form stable micelles. The amphiphilic proteins of the instant disclosure contain a hydrophilic repetitive sequence derived from a naturally disordered protein (e.g., an intrinsically disordered protein (IDP)) and a designed hydrophobic region to allow for self-aggregation. Creating a self-assembling amphiphilic protein from a naturally disordered sequence provides an opportunity for further functionalization that has yet to be realized with methods for preparing amphiphilic proteins that require the use of naturally self-assembling proteins. Furthermore, this disclosure recognizes that genetically encoding a solubility enhancing and cleavable protein group to the amphiphilic proteins described herein provides an efficient method for producing self-assembling proteins in a controlled manner after expression and initial purification that is not achieved by expressing the amphiphilic protein alone.

In some embodiments, the micelles formed from the recombinant proteins described herein have desirable properties that render these micelles suitable for the delivery of a variety of hydrophobic agents in a myriad of applications. Such desirable properties include, but are not limited to, low critical micelle concentration (CMC), pH stability, temperature stability, encapsulation efficiency, size, potential for exterior modification, and biodegradability. Such applications, include but are not limited to, drug delivery, cosmetics, paints and coatings, crop protection, nanoparticle synthesis and catalysis, home and personal care, and cleaning.

The present technology provides compositions comprising an amphiphilic fusion protein comprising a hydrophilic peptide (H₁) fused to a hydrophobic peptide (H₂). In some embodiments the amphiphilic fusion protein comprises a solubilizing moiety (S) and a proteolytic cleavage site (X) fused to the N-terminus of the hydrophilic peptide. In some embodiments, the amphiphilic fusion proteins of the present technology have the general structure shown below:

S—X—H₁—H₂

In some embodiments, the amphiphilic fusion proteins further comprise a cell targeting peptide (T) between the proteolytic cleavage site (X) and the hydrophilic peptide (H₁) and has the general structure shown below:

S—X-T-H₁—H₂

In some embodiments, the amphiphilic fusion proteins, after cleavage of the S—X domains, spontaneously self-assemble to form stable micelles.

In some embodiments the amphiphilic fusion protein comprises a insolubilizing moiety (I) and a chemical cleavage site (X) fused to the N-terminus of the hydrophilic peptide. In some embodiments, the amphiphilic fusion proteins of the present technology have the general structure shown below:

I—X—H₁—H₂.

In some embodiments, the amphiphilic fusion proteins further comprise a cell targeting peptide (T) between the chemical cleavage site (X) and the hydrophilic peptide (H₁) and has the general structure shown below:

I-X-T-H₁—H₂

In some embodiments, the amphiphilic fusion proteins, after cleavage of the I-X domains, spontaneously self-assemble to form stable micelles.

A. Hydrophilic peptides (H₁)

In some embodiments, the hydrophilic peptides of the present technology comprise an intrinsically disordered protein (IDP). In some embodiments, the IDP comprises a polypeptide sequence selected from a human neurofilament protein, San1 protein, Hsp-33 protein, E1A protein, PhD protein, Sic1 protein, WASP protein, p27 protein, CREB protein, PUP protein, LEA protein, or portions or fragments thereof containing intrinsically disordered regions. In some embodiments, the IDP comprises the entire protein or fragments of proteins containing intrinsically disordered peptide regions. In some embodiments, the IDPs of the present technology comprise the human neurofilament polypeptide sequence as set forth in SEQ ID NO: 2, or fragments thereof. In some embodiments, the IDPs of the present technology comprise the human neurofilament polypeptide sequence as set forth in SEQ ID NO: 69, or fragments thereof. Exemplary human neurofilament nucleic acid and polypeptide sequences are provided in Table 1.

TABLE 1
Exemplary human neurofilament nucleic acid and
polypeptide sequences.
Exemplary human neurofilament nucleic acid.
GCCTGGCGTGGCTCCCCGTGGGCAGAGGCCAAGAGTCCAGCGGAAGC
TAAGTCGCCAGCCGAAGTCAAGTCGCCCGCCGTCGCGAAAAGCCCCG
CAGAGGTGAAATCCCCGGCCGAAGTCAAATCGCCGGCAGAAGCGAAA
TCCCCGGCAGAAGCAAAAAGTCCTGCTGAGGTCAAATCGCCAGCAAC
CGTCAAATCCCCTGGAGAGGCAAAATCTCCGGCAGAAGCCAAGTCCC
CTGCCGAAGTGAAGTCACCTGTCGAAGCCAAGTCGCCGGCCGAAGCG
AAGAGCCCAGCGAGCGTGAAAAGTCCTGGTGAGGCTAAGTCCCCGGC
GGAAGCGAAATCTCCAGCGGAAGTAAAGAGTCCGGCCACCGTTAAAT
CCCCGGTAGAGGCCAAAAGCCCTGCGGAAGTTAAATCGCCGGTGACG
GTCAAATCACCCGCGGAAGCGAAGTCCCCGGTGGAGGTGAAATCTCC
GTACTGGTGTGCCTAA (SEQ ID NO: 1)
Human neurofilament polypeptide sequence
(molecular weight 16238.37 Da).
AWRGSPWAEAKSPAEAKSPAEVKSPAVAKSPAEVKSPAEVKSPAEAK
SPAEAKSPAEVKSPATVKSPGEAKSPAEAKSPAEVKSPVEAKSPAEA
KSPASVKSPGEAKSPAEAKSPAEVKSPATVKSPVEAKSPAEVKSPVT
VKSPAEAKSPVEVKSPYWCA (SEQ ID NO: 2)
Human neurofilament polypeptide sequence.
LAEAKSPAEAKSPAEVKSPAVAKSPAEVKSPAEVKSPAEAKSPAEAK
SPAEVKSPATVKSPGEAKSPAEAKSPAEVKSPVEAKSPAEAKSPASV
KSPGEAKSPAEAKSPAEVKSPATVKSPVEAKSPAEVKSPVTVKSPAE
AKSPVEVKSPYWSA (SEQ ID NO: 69)

In some embodiments, the IDPs of the present technology comprise repeats of the sequence (SPAEAK)ₙ (SEQ ID NO: 3), where n is an integer from 2 to 100, or any range in between, such as 2 to 50, or 2 to 25. In some embodiments, n is 25. In some embodiments, the IDPs of the present technology comprise repeats of the sequence (SPAEAR)_{d} (SEQ ID NO: 4), where d is an integer from 2 to 100, or any range in between, such as 2 to 50, or 2 to 25. In some embodiments, d is 25. In some embodiments, the IDP comprises repeats of the sequence (SPAX₁AX₂)ₙ (SEQ ID NO: 53), where X₁ and X₂ are each any charged amino acid and n is an integer from 2 to 100, or any range in between, such as 2 to 50, or 2 to 25. In some embodiments, n is 25.

B. Hydrophobic Peptides (H₂)

In some embodiments, the hydrophobic peptides of the present technology comprise a hydrophobic polypeptide sequence selected from the group consisting of: YGAYAQYVYIYAYWYL (SEQ ID NO: 5), YGAYAQYVYIYAYWYLYAYI (SEQ ID NO: 6), WEAKLAKALAKALAKHLAKALAKALKACEA (SEQ ID NO: 7), YWCCA(X)ₐ (SEQ ID NO: 8) where a is a number of any hydrophobic residue (X), YWXXV_{b}A_{b} (SEQ ID NO: 9) where b is an integer of 3 or greater and X is any hydrophobic residue, and YWA(X)_{c} (SEQ ID NO: 10) where c is a number of any hydrophobic residue (X).

C. Solubilizing Moieties (S)/Insolubilizing Moieties (I)

In some embodiments, the solubilizing moieties of the present technology comprise one or more of a maltose binding protein (MBP) polypeptide sequence, a small ubiquitin-like modifier (SUMO) polypeptide sequence, a glutathione S-transferase (GST) polypeptide sequence, a SlyD polypeptide sequence, a NusA polypeptide sequence, a thioredoxin polypeptide sequence, a ubiquitin polypeptide sequence, or a T7 gene 10 polypeptide sequence. In some embodiments, the solubilizing moiety further comprises a polyhistidine tag (His-tag), such as a 6×His tag. An exemplary nucleic acid sequence for an MBP and its polypeptide sequence are set forth in Table 2A.

TABLE 2A
Exemplary maltose binding protein nucleic
acid and polypeptide sequences.
Exemplary maltose binding protein nucleic acid.
ATGGCCAGCAGCCATCATCATCATCATCACGATTACGATATCCCAAC
GACCGAAAACCTTTACTTCCAGGGATCCAAAATCGAAGAAGGTAAAC
TGGTAATCTGGATTAACGGCGATAAAGGCTATAACGGTCTCGCTGAA
GTCGGTAAGAAATTCGAGAAAGATACCGGAATTAAAGTCACCGTTGA
GCATCCGGATAAACTGGAAGAGAAATTCCCACAGGTTGCGGCAACTG
GCGATGGCCCTGACATTATCTTCTGGGCACACGACCGCTTTGGTGGC
TACGCTCAATCTGGCCTGTTGGCTGAAATCACCCCGGACAAAGCGTT
CCAGGACAAGCTGTATCCGTTTACCTGGGATGCCGTACGTTACAACG
GCAAGCTGATTGCTTACCCGATCGCTGTTGAAGCGTTATCGCTGATT
TATAACAAAGATCTGCTGCCGAACCCGCCAAAAACCTGGGAAGAGAT
CCCGGCGCTGGATAAAGAACTGAAAGCGAAAGGTAAGAGCGCGCTGA
TGTTCAACCTGCAAGAACCGTACTTCACCTGGCCGCTGATTGCTGCT
GACGGGGGTTATGCGTTCAAGTATGAAAACGGCAAGTACGACATTAA
AGACGTGGGCGTGGATAACGCTGGCGCGAAAGCGGGTCTGACCTTCC
TGGTTGACCTGATTAAAAACAAACACATGAATGCAGACACCGATTAC
TCCATCGCAGAAGCTGCCTTTAATAAAGGCGAAACAGCGATGACCAT
CAACGGCCCGTGGGCATGGTCCAACATCGACACCAGCAAAGTGAATT
ATGGTGTAACGGTACTGCCGACCTTCAAGGGTCAACCATCCAAACCG
TTCGTTGGCGTGCTGAGCGCAGGTATTAACGCCGCCAGTCCGAACAA
AGAGCTGGCAAAAGAGTTCCTCGAAAACTATCTGCTGACTGATGAAG
GTCTGGAAGCGGTTAATAAAGACAAACCGCTGGGTGCCGTAGCGCTG
AAGTCTTACGAGGAAGAGTTGGCGAAAGATCCACGTATTGCCGCCAC
TATGGAAAACGCCCAGAAAGGTGAAATCATGCCGAACATCCCGCAGA
TGTCCGCTTTCTGGTATGCCGTGCGTACTGCGGTGATCAACGCCGCC
AGCGGTCGTCAGACTGTCGATGAAGCCCTGAAAGACGCGCAGACTAA
TTCGAGCTCGAACAACAACAACAATAACAATAACAACAACCTCGGGG
CTAGC (SEQ ID NO: 11)
Maltose binding protein polypeptide sequence.
MASSHHHHHHDYDIPTTENLYFQGSKIEEGKLVIWINGDKGYNGLAE
VGKKFEKDTGIKVTVEHPDKLEEKFPQVAATGDGPDIIFWAHDRFGG
YAQSGLLAEITPDKAFQDKLYPFTWDAVRYNGKLIAYPIAVEALSLI
YNKDLLPNPPKTWEEIPALDKELKAKGKSALMFNLQEPYFTWPLIAA
DGGYAFKYENGKYDIKDVGVDNAGAKAGLTFLVDLIKNKHMNADTDY
SIAEAAFNKGETAMTINGPWAWSNIDTSKVNYGVTVLPTFKGQPSKP
FVGVLSAGINAASPNKELAKEFLENYLLTDEGLEAVNKDKPLGAVAL
KSYEEELAKDPRIAATMENAQKGEIMPNIPQMSAFWYAVRTAVINAA
SGRQTVDEALKDAQTNSSSNNNNNNNNNNLGAS
(SEQ ID NO: 12)

In some embodiments, the insolubilizing moieties of the present technology comprise a ketosteroid isomerase (KSI) polypeptide sequence. An exemplary nucleic acid sequence for a KSI and its polypeptide sequence are set forth in Table 2B.

TABLE 2B
Exemplary ketosteroid protein nucleic
acid and polypeptide sequences.
Exemplary ketosteroid isomerase
protein nucleic acid.
atgcataccccggaacatattaccgcggtggtgcagcgctttgtggc
ggcgctgaacgcgggcgatctggatggcattgtggcgctgtttgcgg
atgatgcgaccgtggaagatccggtgggcagcgaaccgcgcagcggc
accgcggcgattcgcgaattttatgcgaacagcctgaaactgccgct
ggcggtggaactgacccaggaagtgcgcgcggtggcgaacgaagcgg
cgtttgcgtttaccgtgagctttgaatatcagggccgcaaaaccgtg
gtggcgccgattgatcattttcgctttaacggcgcgggcaaagtggt
gagcattcgcgcgctgtttggcgaaaaaaacattcatgcgtgccag
(SEQ ID NO: 58)
Ketosteroid isomerase protein
polypeptide sequence.
MHTPEHITAVVQRFVAALNAGDLDGIVALFADDATVEDPVGSEPRSG
TAAIREFYANSLKLPLAVELTQEVRAVANEAAFAFTVSFEYQGRKTV
VAPIDHFRFNGAGKVVSIRALFGEKNIHACQ
(SEQ ID NO: 59)

D. Proteolytic Cleavage Sites/Chemical Cleavage Sites (X)

Exemplary, non-limiting proteolytic cleavage sites comprise a thrombin cleavage site (e.g., LVPR; SEQ ID NO: 13), a tobacco etch virus cleavage site (e.g., ENLYFQ; SEQ ID NO: 14), a 3C cleavage site (e.g., LEVLFQ; SEQ ID NO: 15), an enterokinase cleavage site (e.g., DDDDK; SEQ ID NO: 16), or a Factor Xa cleavage site (e.g., IEGR; SEQ ID NO: 17).

Exemplary, non-limiting chemical cleavage sites comprise a chemical cleavage site selected from a CNBr (cyanogen bromide) cleavage site that cleaves at a methionine residue or a 2-nitro-5-thiocyanobenzoic acid cleavage site that cleaves at a cysteine residue.

E. Cell Targeting Peptides (T)

In some embodiments, the amphiphilic fusion proteins further comprise a cell targeting peptide (T) that can be used to specifically target the amphiphilic fusion proteins or micelles comprising the amphiphilic fusion proteins to a particular cell or tissue. In some embodiments, the cell targeting peptides are useful in methods for delivering hydrophobic cargo to the interior of target cells (e.g., cancer cells, fungal cells, microbial cells).

Thus, in some embodiments, the cell targeting peptide is a cancer cell-targeting peptide selected from the group consisting of a peptide targeting human head and neck solid tumors and having the amino acid sequence TSPLNIHNGQKL (SEQ ID NO: 18), a peptide targeting tumor neovasculature and having the amino acid sequence CGKRK (SEQ ID NO: 19), a peptide targeting breast carcinoma and having the amino acid sequence CGNKRTRGC (SEQ ID NO: 20), a peptide targeting prostate vasculature and having the amino acid sequence SMSIARL (SEQ ID NO: 21), a peptide targeting hepatocellular carcinoma cells and having the amino acid sequence FQHPSFI (SEQ ID NO: 22), a peptide targeting integrin receptor and having the amino acid sequence RGD (SEQ ID NO: 23), a peptide targeting tumor neovasculature and having the amino acid sequence NGR (SEQ ID NO: 24), a peptide targeting endothelial VCAM-1 expressing cells and having the amino acid sequence VHSPNKK (SEQ ID NO: 25), a peptide targeting adenocarcinoma cells and having the amino acid sequence RRPYIL (SEQ ID NO: 26), a peptide targeting various carcinoma and having the amino acid sequence EDYELMDLLAYL (SEQ ID NO: 27), a peptide targeting breast carcinoma and having the amino acid sequence LTVSPWY (SEQ ID NO: 28), and a peptide targeting tumor neovasculature and having the amino acid sequence ATWLPPR (SEQ ID NO: 29).

In some embodiments, the cell targeting peptide comprises a chitin binding domain (CBD) that targets fungal cells.

In some embodiments, the cell targeting peptide comprises an antimicrobial peptide that targets microbes. In some embodiments, the antimicrobial peptide is selected from the group consisting of a dermcidin, an apidaecin, a bactenecin, and a pyrrhocoricin. In some embodiments, the dermcidin is a dermcidin variant selected from the group consisting of DCD-1L comprising the amino acid sequence SSLLEKGLDGAKKAVGGLGKLGKDAVEDLESVGKGAVHDVKDVLDSVL (SEQ ID NO: 30), DCD-1 comprising the amino acid sequence SSLLEKGLDGAKKAVGGLGKLGKDAVEDLESVGKGAVHDVKDVLDSV (SEQ ID NO: 31), and SSL25 comprising the amino acid sequence SSLLEKGLDGAKKAVGGLGKLGKDA (SEQ ID NO: 32). In some embodiments, the apidaecin comprises the amino acid sequence GNNRP(V/I)YIPQPRPPHPR(L/I) (SEQ ID NO: 33). In some embodiments, the bactenecin is bactenecin 5 (Bac 5) or bactenecin 7 (Bac 7). In some embodiments, the pyrrhocoricin comprises the amino acid sequence

(SEQ ID NO: 34)
VDKGSYLPRPTPPRPIYNRN.

F. Recombinant Fusion Protein Nucleic Acid and Amino Acid Sequences

Exemplary IDP-Maltose binding protein (MBP) nucleic acid and amino acid sequences are provided in Table 3A.

TABLE 3A
Exemplary IDP-MBP nucleic acid and
amino acid sequences.
Exemplary IDP-MBP nucleic acid.
ATGGCCAGCAGCCATCATCATCATCATCACGATTACGATATCCCAAC
GACCGAAAACCTTTACTTCCAGGGATCCAAAATCGAAGAAGGTAAAC
TGGTAATCTGGATTAACGGCGATAAAGGCTATAACGGTCTCGCTGAA
GTCGGTAAGAAATTCGAGAAAGATACCGGAATTAAAGTCACCGTTGA
GCATCCGGATAAACTGGAAGAGAAATTCCCACAGGTTGCGGCAACTG
GCGATGGCCCTGACATTATCTTCTGGGCACACGACCGCTTTGGTGGC
TACGCTCAATCTGGCCTGTTGGCTGAAATCACCCCGGACAAAGCGTT
CCAGGACAAGCTGTATCCGTTTACCTGGGATGCCGTACGTTACAACG
GCAAGCTGATTGCTTACCCGATCGCTGTTGAAGCGTTATCGCTGATT
TATAACAAAGATCTGCTGCCGAACCCGCCAAAAACCTGGGAAGAGAT
CCCGGCGCTGGATAAAGAACTGAAAGCGAAAGGTAAGAGCGCGCTGA
TGTTCAACCTGCAAGAACCGTACTTCACCTGGCCGCTGATTGCTGCT
GACGGGGGTTATGCGTTCAAGTATGAAAACGGCAAGTACGACATTAA
AGACGTGGGCGTGGATAACGCTGGCGCGAAAGCGGGTCTGACCTTCC
TGGTTGACCTGATTAAAAACAAACACATGAATGCAGACACCGATTAC
TCCATCGCAGAAGCTGCCTTTAATAAAGGCGAAACAGCGATGACCAT
CAACGGCCCGTGGGCATGGTCCAACATCGACACCAGCAAAGTGAATT
ATGGTGTAACGGTACTGCCGACCTTCAAGGGTCAACCATCCAAACCG
TTCGTTGGCGTGCTGAGCGCAGGTATTAACGCCGCCAGTCCGAACAA
AGAGCTGGCAAAAGAGTTCCTCGAAAACTATCTGCTGACTGATGAAG
GTCTGGAAGCGGTTAATAAAGACAAACCGCTGGGTGCCGTAGCGCTG
AAGTCTTACGAGGAAGAGTTGGCGAAAGATCCACGTATTGCCGCCAC
TATGGAAAACGCCCAGAAAGGTGAAATCATGCCGAACATCCCGCAGA
TGTCCGCTTTCTGGTATGCCGTGCGTACTGCGGTGATCAACGCCGCC
AGCGGTCGTCAGACTGTCGATGAAGCCCTGAAAGACGCGCAGACTAA
TTCGAGCTCGAACAACAACAACAATAACAATAACAACAACCTCGGGG
CTAGCTTAGTTCCTCGTGCCTGGCGTGGCTCCCCGTGGGCAGAGGCC
AAGAGTCCAGCGGAAGCTAAGTCGCCAGCCGAAGTCAAGTCGCCCGC
CGTCGCGAAAAGCCCCGCAGAGGTGAAATCCCCGGCCGAAGTCAAAT
CGCCGGCAGAAGCGAAATCCCCGGCAGAAGCAAAAAGTCCTGCTGAG
GTCAAATCGCCAGCAACCGTCAAATCCCCTGGAGAGGCAAAATCTCC
GGCAGAAGCCAAGTCCCCTGCCGAAGTGAAGTCACCTGTCGAAGCCA
AGTCGCCGGCCGAAGCGAAGAGCCCAGCGAGCGTGAAAAGTCCTGGT
GAGGCTAAGTCCCCGGCGGAAGCGAAATCTCCAGCGGAAGTAAAGAG
TCCGGCCACCGTTAAATCCCCGGTAGAGGCCAAAAGCCCTGCGGAAG
TTAAATCGCCGGTGACGGTCAAATCACCCGCGGAAGCGAAGTCCCCG
GTGGAGGTGAAATCTCCGTACTGGTGTGCCTAA
(SEQ ID NO: 35)
UPPER CASE = MBP nucleic acid
UNDERLINE UPPER CASE = Thrombin cleavage site
BOLD UPPER CASE = IDP nucleic acid
IDP-MBP polypeptide
(molecular weight 61683.50 Da).
MASSHHHHHHDYDIPTTENLYFQGSKIEEGKLVIWINGDKGYNGLAE
VGKKFEKDTGIKVTVEHPDKLEEKFPQVAATGDGPDIIFWAHDRFGG
YAQSGLLAEITPDKAFQDKLYPFTWDAVRYNGKLIAYPIAVEALSLI
YNKDLLPNPPKTWEEIPALDKELKAKGKSALMFNLQEPYFTWPLIAA
DGGYAFKYENGKYDIKDVGVDNAGAKAGLTFLVDLIKNKHMNADTDY
SIAEAAFNKGETAMTINGPWAWSNIDTSKVNYGVTVLPTFKGQPSKP
FVGVLSAGINAASPNKELAKEFLENYLLTDEGLEAVNKDKPLGAVAL
KSYEEELAKDPRIAATMENAQKGEIMPNIPQMSAFWYAVRTAVINAA
SGRQTVDEALKDAQTNSSSNNNNNNNNNNLGASLVPRAWRGSPWAEA
KSPAEAKSPAEVKSPAVAKSPAEVKSPAEVKSPAEAKSPAEAKSPAE
VKSPATVKSPGEAKSPAEAKSPAEVKSPVEAKSPAEAKSPASVKSPG
EAKSPAEAKSPAEVKSPATVKSPVEAKSPAEVKSPVTVKSPAEAKSP
VEVKSPYWCA (SEQ ID NO: 36)
UPPER CASE = MBP polypeptide
UNDERLINE UPPER CASE = Thrombin cleavage site
BOLD UPPER CASE = IDP polypeptide

An exemplary IDP-ketosteroid isomerase protein (KSI) amino acid sequence is provided in Table 3B.

TABLE 3B
Exemplary IDP-KSI amino acid sequence.
IDP-KSI polypeptide
MHTPEHITAVVQRFVAALNAGDLDGIVALFADDATVEDPVGSEPRSG
TAAIREFYANSLKLPLAVELTQEVRAVANEAAFAFTVSFEYQGRKTV
VAPIDHFRFNGAGKVVSIRALFGEKNIHACQMLAEAKSPAEAKSPAE
VKSPAVAKSPAEVKSPAEVKSPAEAKSPAEAKSPAEVKSPATVKSPG
EAKSPAEAKSPAEVKSPVEAKSPAEAKSPASVKSPGEAKSPAEAKSP
AEVKSPATVKSPVEAKSPAEVKSPVTVKSPAEAKSPVEVKSPYWSA
(SEQ ID NO: 60)
UPPER CASE = KSI polypeptide
UNDERLINE UPPER CASE = CNBr cleavage site
BOLD UPPER CASE = IDP polypeptide

Exemplary sequences for amphiphilic fusion proteins of the present technology, comprising a hydrophobic polypeptide sequence fused to a hydrophilic polypeptide sequence, and designated as IDP₁-2Yx2A, IDP₂-2Yx2A, IDP-2Yx3A, and IDP-2Yx4A are set forth in Tables 4A, 4B, 5, and 6 respectively.

TABLE 4A
Exemplary IDP₁-2Yx2A nucleic acid and
amino acid sequences.
2Yx2A-MBP nucleic acid sequence.
ATGGCCAGCAGCCATCATCATCATCATCACGATTACGATATCCCAAC
GACCGAAAACCTTTACTTCCAGGGATCCAAAATCGAAGAAGGTAAAC
TGGTAATCTGGATTAACGGCGATAAAGGCTATAACGGTCTCGCTGAA
GTCGGTAAGAAATTCGAGAAAGATACCGGAATTAAAGTCACCGTTGA
GCATCCGGATAAACTGGAAGAGAAATTCCCACAGGTTGCGGCAACTG
GCGATGGCCCTGACATTATCTTCTGGGCACACGACCGCTTTGGTGGC
TACGCTCAATCTGGCCTGTTGGCTGAAATCACCCCGGACAAAGCGTT
CCAGGACAAGCTGTATCCGTTTACCTGGGATGCCGTACGTTACAACG
GCAAGCTGATTGCTTACCCGATCGCTGTTGAAGCGTTATCGCTGATT
TATAACAAAGATCTGCTGCCGAACCCGCCAAAAACCTGGGAAGAGAT
CCCGGCGCTGGATAAAGAACTGAAAGCGAAAGGTAAGAGCGCGCTGA
TGTTCAACCTGCAAGAACCGTACTTCACCTGGCCGCTGATTGCTGCT
GACGGGGGTTATGCGTTCAAGTATGAAAACGGCAAGTACGACATTAA
AGACGTGGGCGTGGATAACGCTGGCGCGAAAGCGGGTCTGACCTTCC
TGGTTGACCTGATTAAAAACAAACACATGAATGCAGACACCGATTAC
TCCATCGCAGAAGCTGCCTTTAATAAAGGCGAAACAGCGATGACCAT
CAACGGCCCGTGGGCATGGTCCAACATCGACACCAGCAAAGTGAATT
ATGGTGTAACGGTACTGCCGACCTTCAAGGGTCAACCATCCAAACCG
TTCGTTGGCGTGCTGAGCGCAGGTATTAACGCCGCCAGTCCGAACAA
AGAGCTGGCAAAAGAGTTCCTCGAAAACTATCTGCTGACTGATGAAG
GTCTGGAAGCGGTTAATAAAGACAAACCGCTGGGTGCCGTAGCGCTG
AAGTCTTACGAGGAAGAGTTGGCGAAAGATCCACGTATTGCCGCCAC
TATGGAAAACGCCCAGAAAGGTGAAATCATGCCGAACATCCCGCAGA
TGTCCGCTTTCTGGTATGCCGTGCGTACTGCGGTGATCAACGCCGCC
AGCGGTCGTCAGACTGTCGATGAAGCCCTGAAAGACGCGCAGACTAA
TTCGAGCTCGAACAACAACAACAATAACAATAACAACAACCTCGGGG
CTAGCTTAGTTCCTCGTGCCTGGCGTGGCTCCCCGTGGGCAGAGGCC
AAGAGTCCAGCGGAAGCTAAGTCGCCAGCCGAAGTCAAGTCGCCCGC
CGTCGCGAAAAGCCCCGCAGAGGTGAAATCCCCGGCCGAAGTCAAAT
CGCCGGCAGAAGCGAAATCCCCGGCAGAAGCAAAAAGTCCTGCTGAG
GTCAAATCGCCAGCAACCGTCAAATCCCCTGGAGAGGCAAAATCTCC
GGCAGAAGCCAAGTCCCCTGCCGAAGTGAAGTCACCTGTCGAAGCCA
AGTCGCCGGCCGAAGCGAAGAGCCCAGCGAGCGTGAAAAGTCCTGGT
GAGGCTAAGTCCCCGGCGGAAGCGAAATCTCCAGCGGAAGTAAAGAG
TCCGGCCACCGTTAAATCCCCGGTAGAGGCCAAAAGCCCTGCGGAAG
TTAAATCGCCGGTGACGGTCAAATCACCCGCGGAAGCGAAGTCCCCG
GTGGAGGTGAAATCTCCGTACTGGTGTGCCTATGGCGCGTATGCGCA
GTATGTGTATATTTATGCGTATTGGTATCTGTAA
(SEQ ID NO: 37)
UPPER CASE = MBP nucleic acid
UNDERLINE UPPER CASE = Thrombin cleavage site
BOLD UPPER CASE = IDP nucleic acid
UNDERLINED BOLD UPPER CASE = Hydrophobic
polypeptide nucleic acid
2Yx2A-MBP polypeptide sequence
(molecular weight 63604.62 Da).
MASSHHHHHHDYDIPTTENLYFQGSKIEEGKLVIWINGDKGYNGLAE
VGKKFEKDTGIKVTVEHPDKLEEKFPQVAATGDGPDIIFWAHDRFGG
YAQSGLLAEITPDKAFQDKLYPFTWDAVRYNGKLIAYPIAVEALSLI
YNKDLLPNPPKTWEEIPALDKELKAKGKSALMFNLQEPYFTWPLIAA
DGGYAFKYENGKYDIKDVGVDNAGAKAGLTFLVDLIKNKHMNADTDY
SIAEAAFNKGETAMTINGPWAWSNIDTSKVNYGVTVLPTFKGQPSKP
FVGVLSAGINAASPNKELAKEFLENYLLTDEGLEAVNKDKPLGAVAL
KSYEEELAKDPRIAATMENAQKGEIMPNIPQMSAFWYAVRTAVINAA
SGRQTVDEALKDAQTNSSSNNNNNNNNNNLGASLVPRAWRGSPWAEA
KSPAEAKSPAEVKSPAVAKSPAEVKSPAEVKSPAEAKSPAEAKSPAE
VKSPATVKSPGEAKSPAEAKSPAEVKSPVEAKSPAEAKSPASVKSPG
EAKSPAEAKSPAEVKSPATVKSPVEAKSPAEVKSPVTVKSPAEAKSP
VEVKSPYWCAYGAYAQYVYIYAYWYL (SEQ ID NO: 38)
UPPER CASE = MBP polypeptide
UNDERLINE UPPER CASE = Thrombin cleavage site
BOLD UPPER CASE = IDP polypeptide
UNDERLINED BOLD UPPER CASE =
Hydrophobic polypeptide
IDP₁-2Yx2A (molecular weight: 18290.69 Da).
AWRGSPWAEAKSPAEAKSPAEVKSPAVAKSPAEVKSPAEVKSPAEAK
SPAEAKSPAEVKSPATVKSPGEAKSPAEAKSPAEVKSPVEAKSPAEA
KSPASVKSPGEAKSPAEAKSPAEVKSPATVKSPVEAKSPAEVKSPVT
VKSPAEAKSPVEVKSPYWCAYGAYAQYVYIYAYWYL
(SEQ ID NO: 39)
BOLD UPPER CASE = IDP polypeptide
UNDERLINED BOLD UPPER CASE =
Hydrophobic polypeptide

TABLE 4B
Exemplary IDP₂-2Yx2A amino acid sequence.
KSI-IDP-2Yx2A polypeptide sequence.
MHTPEHITAVVQRFVAALNAGDLDGIVALFADDATVEDPVGSEPRSG
TAAIREFYANSLKLPLAVELTQEVRAVANEAAFAFTVSFEYQGRKTV
VAPIDHFRFNGAGKVVSIRALFGEKNIHACQMLAEAKSPAEAKSPAE
VKSPAVAKSPAEVKSPAEVKSPAEAKSPAEAKSPAEVKSPATVKSPG
EAKSPAEAKSPAEVKSPVEAKSPAEAKSPASVKSPGEAKSPAEAKSP
AEVKSPATVKSPVEAKSPAEVKSPVTVKSPAEAKSPVEVKSPYWSAY
GAYAQYVYIYAYWYLM [symbol]
(SEQ ID NO: 61)
UPPER CASE = KSI polypeptide
UNDERLINE UPPER CASE = CNBr cleavage site
BOLD UPPER CASE = IDP polypeptide
UNDERLINED BOLD UPPER CASE =
Hydrophobic polypeptide [symbol]
= His-tag
IDP₂-2Yx2A
LAEAKSPAEAKSPAEVKSPAVAKSPAEVKSPAEVKSPAEAKSPAEAK
SPAEVKSPATVKSPGEAKSPAEAKSPAEVKSPVEAKSPAEAKSPASV
KSPGEAKSPAEAKSPAEVKSPATVKSPVEAKSPAEVKSPVTVKSPAE
AKSPVEVKSPYWSAYGAYAQYVYIYAYWYL
(SEQ ID NO: 56)
BOLD UPPER CASE = IDP polypeptide
UNDERLINED BOLD UPPER CASE =
Hydrophobic polypeptide

TABLE 5
Exemplary IDP-2Yx3A nucleic acid and
amino acid sequences.
2Yx3A-MBP nucleic acid sequence.
ATGGCCAGCAGCCATCATCATCATCATCACGATTACGATATCCCAAC
GACCGAAAACCTTTACTTCCAGGGATCCAAAATCGAAGAAGGTAAAC
TGGTAATCTGGATTAACGGCGATAAAGGCTATAACGGTCTCGCTGAA
GTCGGTAAGAAATTCGAGAAAGATACCGGAATTAAAGTCACCGTTGA
GCATCCGGATAAACTGGAAGAGAAATTCCCACAGGTTGCGGCAACTG
GCGATGGCCCTGACATTATCTTCTGGGCACACGACCGCTTTGGTGGC
TACGCTCAATCTGGCCTGTTGGCTGAAATCACCCCGGACAAAGCGTT
CCAGGACAAGCTGTATCCGTTTACCTGGGATGCCGTACGTTACAACG
GCAAGCTGATTGCTTACCCGATCGCTGTTGAAGCGTTATCGCTGATT
TATAACAAAGATCTGCTGCCGAACCCGCCAAAAACCTGGGAAGAGAT
CCCGGCGCTGGATAAAGAACTGAAAGCGAAAGGTAAGAGCGCGCTGA
TGTTCAACCTGCAAGAACCGTACTTCACCTGGCCGCTGATTGCTGCT
GACGGGGGTTATGCGTTCAAGTATGAAAACGGCAAGTACGACATTAA
AGACGTGGGCGTGGATAACGCTGGCGCGAAAGCGGGTCTGACCTTCC
TGGTTGACCTGATTAAAAACAAACACATGAATGCAGACACCGATTAC
TCCATCGCAGAAGCTGCCTTTAATAAAGGCGAAACAGCGATGACCAT
CAACGGCCCGTGGGCATGGTCCAACATCGACACCAGCAAAGTGAATT
ATGGTGTAACGGTACTGCCGACCTTCAAGGGTCAACCATCCAAACCG
TTCGTTGGCGTGCTGAGCGCAGGTATTAACGCCGCCAGTCCGAACAA
AGAGCTGGCAAAAGAGTTCCTCGAAAACTATCTGCTGACTGATGAAG
GTCTGGAAGCGGTTAATAAAGACAAACCGCTGGGTGCCGTAGCGCTG
AAGTCTTACGAGGAAGAGTTGGCGAAAGATCCACGTATTGCCGCCAC
TATGGAAAACGCCCAGAAAGGTGAAATCATGCCGAACATCCCGCAGA
TGTCCGCTTTCTGGTATGCCGTGCGTACTGCGGTGATCAACGCCGCC
AGCGGTCGTCAGACTGTCGATGAAGCCCTGAAAGACGCGCAGACTAA
TTCGAGCTCGAACAACAACAACAATAACAATAACAACAACCTCGGGG
CTAGCTTAGTTCCTCGTGCCTGGCGTGGCTCCCCGTGGGCAGAGGCC
AAGAGTCCAGCGGAAGCTAAGTCGCCAGCCGAAGTCAAGTCGCCCGC
CGTCGCGAAAAGCCCCGCAGAGGTGAAATCCCCGGCCGAAGTCAAAT
CGCCGGCAGAAGCGAAATCCCCGGCAGAAGCAAAAAGTCCTGCTGAG
GTCAAATCGCCAGCAACCGTCAAATCCCCTGGAGAGGCAAAATCTCC
GGCAGAAGCCAAGTCCCCTGCCGAAGTGAAGTCACCTGTCGAAGCCA
AGTCGCCGGCCGAAGCGAAGAGCCCAGCGAGCGTGAAAAGTCCTGGT
GAGGCTAAGTCCCCGGCGGAAGCGAAATCTCCAGCGGAAGTAAAGAG
TCCGGCCACCGTTAAATCCCCGGTAGAGGCCAAAAGCCCTGCGGAAG
TTAAATCGCCGGTGACGGTCAAATCACCCGCGGAAGCGAAGTCCCCG
GTGGAGGTGAAATCTCCGTACTGGTGTGCCTATGGCGCGTATGCGCA
GTATGTGTATATTTATGCGTATTGGTATCTGTATGCTTATATTTAA
(SEQ ID NO: 40)
UPPER CASE = MBP nucleic acid
UNDERLINE UPPER CASE = Thrombin cleavage site
BOLD UPPER CASE = IDP nucleic acid
UNDERLINED BOLD UPPER CASE = Hydrophobic
polypeptide nucleic acid
2Yx3A-MBP polypeptide sequence
(molecular weight 64115.21 Da)
MASSHHHHHHDYDIPTTENLYFQGSKIEEGKLVIWINGDKGYNGLAE
VGKKFEKDTGIKVTVEHPDKLEEKFPQVAATGDGPDIIFWAHDRFGG
YAQSGLLAEITPDKAFQDKLYPFTWDAVRYNGKLIAYPIAVEALSLI
YNKDLLPNPPKTWEEIPALDKELKAKGKSALMFNLQEPYFTWPLIAA
DGGYAFKYENGKYDIKDVGVDNAGAKAGLTFLVDLIKNKHMNADTDY
SIAEAAFNKGETAMTINGPWAWSNIDTSKVNYGVTVLPTFKGQPSKP
FVGVLSAGINAASPNKELAKEFLENYLLTDEGLEAVNKDKPLGAVAL
KSYEEELAKDPRIAATMENAQKGEIMPNIPQMSAFWYAVRTAVINAA
SGRQTVDEALKDAQTNSSSNNNNNNNNNNLGASLVPRAWRGSPWAEA
KSPAEAKSPAEVKSPAVAKSPAEVKSPAEVKSPAEAKSPAEAKSPAE
VKSPATVKSPGEAKSPAEAKSPAEVKSPVEAKSPAEAKSPASVKSPG
EAKSPAEAKSPAEVKSPATVKSPVEAKSPAEVKSPVTVKSPAEAKSP
VEVKSPYWCAYGAYAQYVYIYAYWYLYAYI (SEQ ID NO: 41)
UPPER CASE = MBP polypeptide
UNDERLINE UPPER CASE = Thrombin cleavage site
BOLD UPPER CASE = IDP polypeptide
UNDERLINED BOLD UPPER CASE =
Hydrophobic polypeptide
IDP-2Yx3A (molecular weight: 18801.28 Da).
AWRGSPWAEAKSPAEAKSPAEVKSPAVAKSPAEVKSPAEVKSPAEAK
SPAEAKSPAEVKSPATVKSPGEAKSPAEAKSPAEVKSPVEAKSPAEA
KSPASVKSPGEAKSPAEAKSPAEVKSPATVKSPVEAKSPAEVKSPVT
VKSPAEAKSPVEVKSPYWCAYGAYAQYVYIYAYWYLYAYI
(SEQ ID NO: 42)
BOLD UPPER CASE = IDP polypeptide
UNDERLINED BOLD UPPER CASE =
Hydrophobic polypeptide

TABLE 6
Exemplary IDP-2Yx4A amino acid sequence.
IDP-2Yx4A
AWRGSPWAEAKSPAEAKSPAEVKSPAVAKSPAEVKSPAEVKSPAEAK
SPAEAKSPAEVKSPATVKSPGEAKSPAEAKSPAEVKSPVEAKSPAEA
KSPASVKSPGEAKSPAEAKSPAEVKSPATVKSPVEAKSPAEVKSPVT
VKSPAEAKSPVEVKSPYWCAYGAYAQYVYIYAYWYLYAYIAVAL
(SEQ ID NO: 57)
BOLD UPPER CASE = IDP polypeptide
UNDERLINED BOLD UPPER CASE =
Hydrophobic polypeptide

### III. Synthesis

The present section provides a general description of the synthesis, formation and use of the amphiphilic fusion proteins and micellar compositions as described herein. Plasmids encoding the 2Yx2A, 2Yx3A, or 2Yx4A amphiphilic fusion proteins of the present technology are prepared according to the methods outlined in the Examples.

Any suitable expression system for producing the amphiphilic fusion proteins may be employed. In some embodiments, a cell-free system is used for the production of the amphiphilic fusion proteins. In some embodiments, a host cell is transformed with the expression vectors of the present technology. In some embodiments, the host cell is any eukaryotic, prokaryotic, or archaea cell. In some embodiments, the host cell is a yeast, bacterial, cyanobacteria, insect, plant, or mammalian cell. In some embodiments, the host cell is E. coli.

In some embodiments, the present disclosure relates to cell cultures comprising the host cells transformed with the expression vectors comprising chimeric nucleic acids encoding the amphiphilic fusion proteins of the present technology.

The expressed fusion proteins are then digested with a protease (e.g., thrombin) to remove the solubilizing moiety and may then be purified by any suitable means known in the art. Non-limiting purification methods are further described in the Examples.

Also provided herein in one aspect is a method of producing an amphiphilic fusion protein that spontaneously self-assembles to form a stable micelle, the method comprising: (a) introducing into a host cell an expression vector comprising a chimeric nucleic acid construct comprising, in the 5′ to 3′ direction, a promoter suitable for directing expression in a host cell operably linked to a nucleic acid sequence encoding an amphiphilic fusion protein having Formula (I): S—X—H₁—H₂, wherein S— is a solubilizing moiety, —X— is a peptide sequence comprising a proteolytic cleavage site, —H₁— is a hydrophilic peptide, and —H₂ is a hydrophobic peptide; (b) growing the host cell under conditions that allow for expression of the chimeric nucleic acid to produce the amphiphilic fusion protein; (c) purifying the amphiphilic fusion protein; and (d) contacting the amphiphilic fusion protein with a protease to provide an amphiphilic fusion protein having Formula (II): H₁—H₂. In some embodiments, the chimeric nucleic acid construct of part (a) encodes an amphiphilic fusion protein further comprising a cell targeting peptide (-T-) between the —X— and the —H₁—, such that the amphiphilic fusion protein has Formula (III): S—X-T-H₁—H₂, and such that after part (d) the amphiphilic fusion protein has Formula (IV): T-H₁—H₂.

The expressed fusion proteins are then digested with reagent to induce chemical cleavage (e.g., CNBr) to remove the insolubilizing moiety and may then be purified by any suitable means known in the art. Non-limiting purification methods are further described in the Examples.

Also provided herein in one aspect is a method of producing an amphiphilic fusion protein that spontaneously self-assembles to form a stable micelle, the method comprising: (a) introducing into a host cell an expression vector comprising a chimeric nucleic acid construct comprising, in the 5′ to 3′ direction, a promoter suitable for directing expression in a host cell operably linked to a nucleic acid sequence encoding an amphiphilic fusion protein having Formula (I): I—X—H₁—H₂, wherein I— is an insolubilizing moiety, —X— is a peptide sequence comprising a chemical cleavage site, —H₁— is a hydrophilic peptide, and —H₂ is a hydrophobic peptide; (b) growing the host cell under conditions that allow for expression of the chimeric nucleic acid to produce the amphiphilic fusion protein; (c)purifying the amphiphilic fusion protein; and (d) contacting the amphiphilic fusion protein with a reagent to induce chemical cleavage to provide an amphiphilic fusion protein having Formula (II): H₁—H₂. In some embodiments, the chimeric nucleic acid construct of part (a) encodes an amphiphilic fusion protein further comprising a cell targeting peptide (-T-) between the —X— and the —H₁—, such that the amphiphilic fusion protein has Formula (III): I-X-T-H₁—H₂, and such that after part (d) the amphiphilic fusion protein has Formula (IV): T-H₁—H₂.

### IV. Micelle Characteristics

Provided in another aspect are micelles comprising any one of the amphiphilic fusion proteins described herein. In some embodiments, the amphiphilic fusion protein is characterized by a hydrophilic peptide (H₁); and a hydrophobic peptide (H₂).

In some embodiments, the micelle described herein has a low critical micelle concentration (CMC). In some embodiments, the CMC of the amphiphilic fusion protein in water is greater than about 10 μM at a physiological pH of about 7.4. In some embodiments, the CMC of the amphiphilic fusion protein in water is less than about 20 μM at a physiological pH of about 7.4. In some embodiments, the CMC of the amphiphilic fusion protein in water is from about 10 μM to about 20 μM at a physiological pH of about 7.4. In some embodiments, the CMC of the amphiphilic fusion protein in water is about 10 μM, about 11 μM, about 12 μM, about 13 μM, about 14 μM, about 15 μM, about 16 μM, about 17 μM, about 18 μM, about 19 μM, or about 20 μM at a physiological pH of about 7.4.

In some embodiments, the micelle described herein has a diameter from about 20 nm to about 40 nm. In some embodiments, the micelle described herein has a diameter greater than about 20 nm. In some embodiments, the micelle described herein has a diameter less than about 40 nm. In some embodiments, the micelle described herein has a diameter of about 20 nm, about 21 nm, about 22 nm, about 23 nm, about 24 nm, about 25 nm, about 26 nm, about 27 nm, about 28 nm, about 29 nm, about 30 nm, about 31 nm, about 32 nm, about 33 nm, about 34 nm, about 35 nm, about 36 nm, about 37 nm, about 38 nm, about 39 nm, or about 40 nm. In some embodiments, the micelle described herein has a diameter of about 27 nm.

In some embodiments, the micelle described herein is pH stable. In some embodiments, the micelle is stable at a pH from about 2.0 to about 10.0. In some embodiments, the micelle is stable at a pH greater than about 2.0. In some embodiments, the micelle is stable at a pH less than about 10.0. In some embodiments, the micelle is stable at a pH of about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5, or to about 10.0.

In some embodiments, the micelle described herein is temperature stable. In some embodiments, the micelle is stable at a temperature from about 25° C. to about 70° C. In some embodiments, the micelle is stable at a temperature greater than about 25° C. In some embodiments, the micelle is stable at a temperature less than about 70° C. In some embodiments, the micelle is stable at a temperature of about 25° C., about 30° C., about 35° C., about 40° C., about 45° C., about 50° C., about 55° C., about 60° C., about 65° C., about 70° C., or about 75° C.

In some embodiments, the micelle further contains a fluorescent dye. In some embodiments, the fluorescent dye is covalently attached to the hydrophilic peptide (H₁). In some embodiments, the fluorescent dye is covalently attached to the hydrophobic peptide (H₂). In some embodiments, the fluorescent dye is fluorescein or rhodamine.

In some embodiments, the micelle has a core-shell structure. In some embodiments, the micelle has a shell diameter from about 40 nm to about 75 nm. In some embodiments, the micelle has a shell diameter greater than about 40 nm. In some embodiments, the micelle has a shell diameter less than about 75 nm. In some embodiments, the micelle has a shell diameter of about 40 nm, about 45 nm, about 50 nm, about 55 nm, about 60 nm, about 65 nm, about 70 nm, or about 75 nm. In some embodiments, the micelle has a core diameter from about 25 nm to about 45 nm. In some embodiments, the micelle has a core diameter greater than about 25 nm. In some embodiments, the micelle has a core diameter less than about 45 nm. In some embodiments, the micelle has a core diameter of about 25 nm, about 30 nm, about 35 nm, about 40, or about 45 nm. In some embodiments, the micelle has a shell thickness from about 5 nm to about 20 nm. In some embodiments, the micelle has a shell thickness of greater than about 5 nm. In some embodiments, the micelle has a shell thickness of less than about 20 nm. In some embodiments, the micelle has a shell thickness of about 5 nm, about 10 nm, about 15 nm, or about 20 nm.

In some embodiments, the micelle further contains a hydrophobic cargo. In some embodiments, the hydrophobic cargo is a drug, a fungicide, a protein, a nucleic acid, a hormone, a receptor, a diagnostic agent, an imaging agent, a metal complex, a silicone oil, a triglyceride, or a combination thereof. In some embodiments, the hydrophobic cargo is a drug. In some embodiments, the hydrophobic cargo is a fungicide. In some embodiments, the hydrophobic cargo is a protein. In some embodiments, the hydrophobic cargo is a nucleic acid. In some embodiments, the hydrophobic cargo is a hormone. In some embodiments, the hydrophobic cargo is a receptor. In some embodiments, the hydrophobic cargo is a diagnostic agent. In some embodiments, the hydrophobic cargo is an imaging agent. In some embodiments, the hydrophobic cargo is a metal complex. In some embodiments, the hydrophobic cargo is a silicone oil. In some embodiments, the hydrophobic cargo is a triglyceride.

### V. Pharmaceutical Compositions

In another aspect, compositions, e.g., “pharmaceutical compositions” are provided comprising and an effective amount of a micelle described herein, a hydrophobic cargo, and/or a therapeutically active agent. In some embodiments, the composition further includes at least one pharmaceutically acceptable excipient.

Pharmaceutical compositions comprising a micelle described herein, a hydrophobic cargo, and/or a therapeutically active agent can be formulated for different routes of administration, including intravenous, intraarterial, pulmonary, rectal, nasal, vaginal, lingual, intramuscular, intraperitoneal, intracutaneous, transdermal, intracranial, subcutaneous and oral routes. Other dosage forms include tablets, capsules, pills, powders, aerosols, suppositories, parenterals, and oral liquids, including suspensions, solutions and emulsions. All dosage forms may be prepared using methods that are standard in the art (see e.g., Remington's Pharmaceutical Sciences, 16^{th} ed., A. Oslo editor, Easton Pa. 1980).

In some embodiments, the micelle described herein, hydrophobic cargo, and/or therapeutically active agent are formulated in conjunction with appropriate salts and buffers to render delivery of the compositions in a stable manner to allow for uptake by target cells. Buffers also are employed when the micelle described herein, hydrophobic cargo, and/or therapeutically active agent are introduced into a patient. In some embodiments, an aqueous composition is used, comprising an effective amount of the micelle, hydrophobic cargo, and/or therapeutically active agent, which are dispersed in a pharmaceutically acceptable carrier or excipient an aqueous medium. As used herein, “pharmaceutically acceptable carrier” includes any and all solvents, dispersion media, coatings, antibacterial and antifungal agents, isotonic and absorption delaying agents and the like. Sterile phosphate-buffered saline is one example of a pharmaceutically suitable excipient. Other suitable carriers and excipients are well-known to those in the art, see, for example, Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

The micelle as described herein, hydrophobic cargo, and/or therapeutically active agent may be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

### VI. Methods of Use

Also provided is an effective method of using the amphiphilic fusion proteins and micellar compositions described herein for delivering hydrophobic cargo and/or therapeutically active agents to the interior of target cells (e.g., cancer cells, fungal cells, microbial cells). Thus, in some embodiments, methods of therapy are provided that comprise or require delivery of hydrophobic cargo and/or therapeutically active agents into a cell. In some embodiments, the hydrophobic cargo and/or therapeutically active agent is a chemotherapeutic drug, e.g., doxorubicin.

In another aspect, a method for treating cancer in a subject is provided, where the method comprises administering to the subject an effective amount of a composition comprising any of the micelles described herein and a therapeutically active agent (e.g., a chemotherapeutic drug). In some embodiments, examples of chemotherapeutic drugs include, but are not limited to, doxorubicin, paclitaxel, and rapamycin. In some embodiments, the therapeutically active agent is a steroidal drug, including, but not limited to, hydrocortisone, testosterone, progesterone, 17β-estradiol, or levonorgestrel. In some embodiments, the micelles of the present technology may be used in methods for delivering to target cells or tissues drugs that are otherwise encapsulated in polymer nanoparticles for efficient delivery including, but not limited to, risperidone, minocycline hydrochloride, or bromocriptine. In some embodiments, the micelles of the present technology are useful in methods for delivering to target cells or tissues imaging agents including, but not limited to, fluorescent dyes, PET probes, and MRI contrast agents.

The dosage of an administered micelle described herein, and hydrophobic cargo, and/or a therapeutically active agent for humans will vary depending upon such factors as the patient's age, weight, height, sex, general medical condition and previous medical history.

In some embodiments, methods and compositions are provided for the treatment of cancer. Cell proliferative disorders, or cancers, contemplated to be treatable with the methods include, but are not limited to, human head and neck solid tumors, breast carcinoma, prostate carcinoma, hepatocellular carcinoma, adenocarcinomas. In some embodiments, the method is used to inhibit growth, progression, and/or metastasis of cancers, in particular those listed above.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims.

### Example 1: Preparation of Fusion Proteins

This example describes the preparation of exemplary fusion proteins described herein.

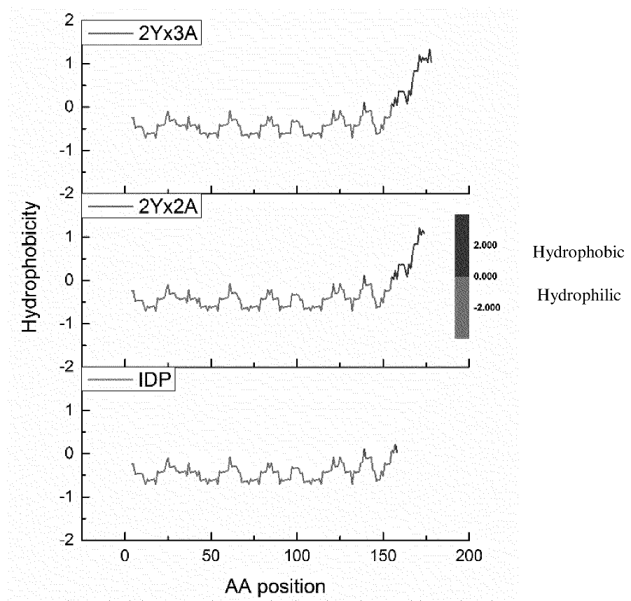

Design of Sequences. The intrinsically disordered sequence derived from the neurofilament heavy arm side chain is a naturally stimulus responsive sequence that flares out around the head domain, giving rise to a cylindrical brush structure. Due to the interesting properties of this highly charged and repetitive sequence, methods were developed to express this portion of the protein with increasing hydrophobic appendages in attempt to create an intrinsically disordered protein that can self-assemble around a genetically encoded hydrophobic sequence (FIG. 1).

Two main challenges arise with recombinantly expressed proteins such as these:
1. A high propensity for protease degradation of the disordered region results in truncation and heterogeneity; and
2. Aggregation/self-assembly of proteins in vivo resulting in truncations due to premature ribosome departure, toxicity to host cells, degradation, or shuttling to inclusion bodies.

To address these issues, the constructs were expressed with an N-terminal Maltose Binding Protein (MBP) and a 6×His tag that could then be cleaved from the protein of interest by inserting a thrombin cleavage site between the two proteins. MBP serves to enhance the solubility of the protein constructs during expression and initial steps of purification, allowing for normal protein expression handling techniques. MBP also increases expression yields of constructs it is appended to which is also beneficial for production purposes.

Construction of Plasmids. (a) IDP-MBP plasmid: The inherent repetitive sequence of IDP made the synthesis of one contiguous gene block impossible. Instead, two gene blocks (gBlocks: IDT Technologies) had to be synthesized (IDP-1 and IDP2; see below for respective sequences) with a 32 bp consensus sequence that allows for Gibson assembly. A 100 ng sample of each gBlock and 10 μl of 2× Gibson master mix (ref. Gibson, D. G., Young, L., et al. Nat. Methods. 2009, 6, 343-345) was adjusted with water to a volume of 20 μL and was incubated at 50° C. for 60 min. After DNA clean up with QiaQuick (Qiagen), the assembly product was PCR amplified (VENT polymerase from NEB, Tm=61° C.) with forward and reverse primers: 5′-ATA ATA GCT AGC TTA GTT CCT CGT GCC TGG CGT G-3′ (SEQ ID NO: 43) and 5′-TAT TAT CTC GAG CTA TTA GGC ACA CCA GTA CGG AGA TTT C-3′ (SEQ ID NO: 44). The IDT insert contained NheI and XhoI restriction sites, that were double digested, heat inactivated at 80° C. for 5 min and ligated (QuickLigase, NEB) with a 5′-terminal MBP pSKB3 vector. Plating on Kanamycin agar plates yielded individual colonies that were cultured, DNA purified (NucleoSpin, MacheryNagel) and sequenced (Quintara BioSciences).

gBlock IDP-1:
(SEQ ID NO: 45)
ATAATAGCTAGCTTAGTTCCTCGTGCCTGGCGTGGCTCCCCGTGGGC
AGAGGCCAAGAGTCCAGCGGAAGCTAAGTCGCCAGCCGAAGTCAAGT
CGCCCGCCGTCGCGAAAAGCCCCGCAGAGGTGAAATCCCCGGCCGAA
GTCAAATCGCCGGCAGAAGCGAAATCCCCGGCAGAAGCAAAAAGTCC
TGCTGAGGTCAAATCGCCAGCAACCGTCAAATCCCCTGGAGAGGCAA
AATCTCCGGCAGAAGCCAAGTCCCCTGCCGAAGTGAAGTCAC
gBlock IDP-2:
(SEQ ID NO: 46)
AGAAGCCAAGTCCCCTGCCGAAGTGAAGTCACCTGTCGAAGCCAAGT
CGCCGGCCGAAGCGAAGAGCCCAGCGAGCGTGAAAAGTCCTGGTGAG
GCTAAGTCCCCGGCGGAAGCGAAATCTCCAGCGGAAGTAAAGAGTCC
GGCCACCGTTAAATCCCCGGTAGAGGCCAAAAGCCCTGCGGAAGTTA
AATCGCCGGTGACGGTCAAATCACCCGCGGAAGCGAAGTCCCCGGTG
GAGGTGAAATCTCCGTACTGGTGTGCCTAATAGCTCGAGATAATA
Underlined: Consensus sequence used for
Gibson Assembly

(b) 2Y-MBP plasmid: Overhang PCR was performed on the MBP-IDP plasmid constructed in (a). The forward primer extended the sequence with a Bsa1 cut site while the reverse primer extended the sequence with the desired hydrophobic portion and a Bsa1 cut site to allow for incorporation into our plasmid by golden gate assembly. The amplified sequence was run on a 1% agarose gel and confirmed to be of the approximate length. The PCR product was extracted and purified. To perform the golden gate assembly, the 2Y PCR product was incubated with our golden gate plasmid, Bsa1, NEB ligase buffer, and ligase enzyme and cycled 25 times. After ligation plasmids were transformed into chemically competent cells and plated on Kanamycin LB agar plates at 37° C. overnight. When the agar plate was exposed to UV light, white colonies were selected (green indicating no excision of GFP by Bsa1) and grown in 10 mL of LB media at 37° C. overnight. Plasmid DNA was subsequently purified (NucleoSpin, MacheryNagel) and sequenced (Quintara BioSciences).

Forward primer:
(SEQ ID NO: 47)
5′ AGG TCT CTC ATG GCC AGC AGC CAT CAT 3′
Reverse primer:
(SEQ ID NO: 48)
5′ TGG TCT CGT TTA CAC ATA CTG CGC ATA CGC GCC
ATA GGC ACA CCA GTA CGG AGA TTT CA 3′
Underlined: BsaI cut site

(c) 2Yx2A plasmid construction: Overhang PCR was performed on 2Y-MBP plasmid constructed in (b). The forward primer extended the sequence with a Bsa1 cut site while the reverse primer extended the sequence with a hydrophobic portion and a Bsa1 cut site to allow for incorporation into our plasmid by golden gate assembly. Following the same procedure as in (b) the 2Yx2A plasmid was purified and sequenced.

Forward primer:
(SEQ ID NO: 49)
5′ AGG TCT CTC ATG GCC AGC AGC CAT CAT 3′
Reverse primer:
(SEQ ID NO: 50)
5′ TGG TCT CGT TTA AAT ATA AGC ATA CAG ATA CCA
ATA CGC ATA AAT ATA CAC ATA CTG CG 3′
Underlined: BsaI cut site

(d) 2Yx3A plasmid construction: Overhang PCR containing was performed on 2Yx2A plasmid constructed in (c). The forward primer extended the sequence with a Bsa1 cut site while the reverse primer extended the sequence with a hydrophobic portion and a Bsa1 cut site to allow for incorporation into our plasmid by golden gate assembly. Following the same procedure as in (c) the 2Yx3A plasmid was purified and sequenced.

Forward primer:
(SEQ ID NO: 51)
5′ AGG TCT CTC ATG GCC AGC AGC CAT CAT 3′
Reverse primer:
(SEQ ID NO: 52)
5′ TGG TCT CGT TTA AAT ATA AGC ATA CAG ATA CCA
ATA CGC ATA AAT ATA CAC ATA CTG CG 3′
Underlined: BsaI cut site

Expression of MBP-IDP. (a) Plasmids were transformed into E. coli BL21 (DE3) competent cells. Starter cultures (20 ml LB, 50 mg/L Kanamycin) were grown from single colonies, grown overnight at 37° C., and used to inoculate 1 L of TB media (50 mg/L Kanamycin). Cultures were grown to an OD ˜0.5, cooled for 20 min at 25° C., induced with 0.5 mM IPTG, and expressed overnight (˜18 hours) at 25° C. Cells were harvested by centrifugation for 15 min at 4,000 rcf (g) at 4° C.

Purification of MBP-IDP fusion protein. The pellet was transferred to a 50 ml Falcon tube in PBS buffer, and spun down for 10 min at 4000 rcf (g). The resulting pellet (˜5 g) was lysed in 30 ml of lysis buffer (20 mM HEPES, pH=7.5, 300 mM NaCl, 10 mM imidazole=buffer A) supplemented with one tablet of EDTA-free SigmaFast Protease Inhibitor (Sigma Aldrich), 2 mM PMSF, and 10 mg lysozyme. The resuspended sample was lysed with an Avestin C3 homogenizer followed by 20 min of centrifugation at 24,000 rcf (g) at 4° C. The supernatant was filtered through a 40 μm Steriflip filter (Millipore), and loaded onto a 5 ml NiNTA column (Protino, Machery Nagel) connected to an Akta purifier that was pre-equilibrated with buffer A. The column was washed with 50 ml (10 CV) of 20 mM HEPES (pH=7.5), 300 mM NaCl, 10 mM imidazole, 10 mM βMe. The protein was eluted with 20 mM HEPES (pH=7.5), 300 mM NaCl, 250 mM imidazole, 10 mM βMe. Imidazole was removed by exchanging against 20 mM HEPES (pH=7.5), 100 mM NaCl with a 10DG desalting column (BioRad), and subsequently digested with 1 mg of thrombin protease (high purity from Bovine, MP Biomedicals). Complete digestion was achieved at room temperature after 1 hour as confirmed by LC/MS (ESI/TOF). The protein mixture was diluted with salt-free HEPES buffer (20 mM, pH=7.5) to 50 ml ([NaCl]˜5 mM) before loading it onto a 1 ml HiTrap Q HP cation exchange column connected to an Akta purifier (GE Healthcare). The column was pre-equilibrated with 20 mM HEPES, 10 mM βMe, washed with 10 ml (10 CV) of 20 mM HEPES (pH=7.5), 10 mM βMe and eluted with a gradient from 0-1M NaCl (50 ml total volume). Monomeric IDP eluted at around 200 mM NaCl. Without addition of reducing agent, significant dimeric formation that elutes with little retention at the tail end of the loading step was observed. The protein was eluted with 20 mM HEPES (pH=7.5), 300 mM NaCl, 250 mM imidazole, 10 mM βMe. The final IDP sample was obtained with a final desalting column (10DG, BioRad) to obtain the protein at a final concentration of 270 μM in 20 mM HEPES (pH=7.5), 50 mM NaCl. The protein was >95% pure by SDS-PAGE and LCMS (ESI-TOF; Agilent). The purified protein was flash frozen with liquid N2 in 20 μl aliquots.

Expression and Purification of 2Yx2A-MBP. (b) Expression: Plasmids were transformed into E. coli Rosetta 2 plys competent cells. Starter cultures (20 ml LB, 50 mg/L Kanamycin) were grown from single colonies, grown overnight at 37° C., and used to inoculate 1 L of TB media (50 mg/L Kanamycin). Cultures were grown to an OD ˜0.5, cooled for 20 min at 16° C., induced with 0.1 mM IPTG, and expressed (˜6 hours) at 16° C. Cells were harvested by centrifugation for 15 min at 4,000 rcf (g) at 4° C.

(c) Purification of MBP-2Yx2A fusion protein: The pellet was transferred to a 50 ml Falcon tube in PBS buffer, and spun down for 10 min at 4000 rcf (g). The resulting pellet (˜5 g) was lysed in 30 ml of lysis buffer (20 mM HEPES, pH=7.5, 300 mM NaCl, 10 mM imidazole=buffer A) supplemented with one tablet of EDTA-free SigmaFast Protease Inhibitor (Sigma Aldrich), 2 mM PMSF, and 10 mg lysozyme. The resuspended sample was lysed by sonication (amplitude 50%, 2:4 seconds on off for 10 minutes) followed by 20 min of centrifugation at 24,000 rcf (g) at 4° C. The supernatant was filtered through a 40 μm Steriflip filter (Millipore), and loaded onto a 5 ml NiNTA column (Protino, Machery Nagel) connected to an Akta purifier that was pre-equilibrated with buffer A. The column was washed with 50 ml (10 CV) of 20 mM HEPES (pH=7.5), 300 mM NaCl, 10 mM imidazole. The protein was eluted with 20 mM HEPES (pH=7.5), 300 mM NaCl, 250 mM imidazole. Imidazole was removed by spin concentration with 20 mM HEPES (pH=7.5), 100 mM NaCl. MBP-2Yx2/3A was subsequently digested with 1 mg of thrombin protease (high purity from Bovine, MP Biomedicals). Complete digestion was achieved at room temperature after 1 hour as confirmed by LC/MS. Then either ion exchange or Biotage HPLC purification could be used to remove the residual MBP.

(d) Ion exchange: The protein mixture was diluted with salt-free HEPES buffer (20 mM, pH=7.5) and 8M urea to 50 ml ([NaCl]˜5 mM) before loading it onto a 1 ml HiTrap SP HP cation exchange column connected to an Akta purifier (GE Healthcare). The column was pre-equilibrated with 20 mM HEPES, 10 mM βMe, washed with 10 ml (10 CV) of 20 mM HEPES (pH=7.5) and eluted with a gradient from 0-1 M NaCl (100 ml total volume). 2Yx2A eluted at around 500 mM NaCl. The final 2Yx2A sample was dialyzed against 100 mM PB pH 5.5. Gel analysis shows 75% purity.

(e) Biotage HPLC: 10% acetonitrile (ACN) is added to the crude protein mixture which is then loaded onto a 10 g C18 Biotage SNAP Bio 300A reversed phase column that has been equilibrated with 10% ACN in H₂O+0.1% TFA. The column was run over 14 minutes to 100% ACN with the desired product eluting around 40% ACN. The fractions containing 2Yx2A were analyzed by LC/MS (ESI/TOF) for purity. 100% pure fractions were collected and lyophilized to dryness resulting in a white powder. Gel analysis shows >95% purity.

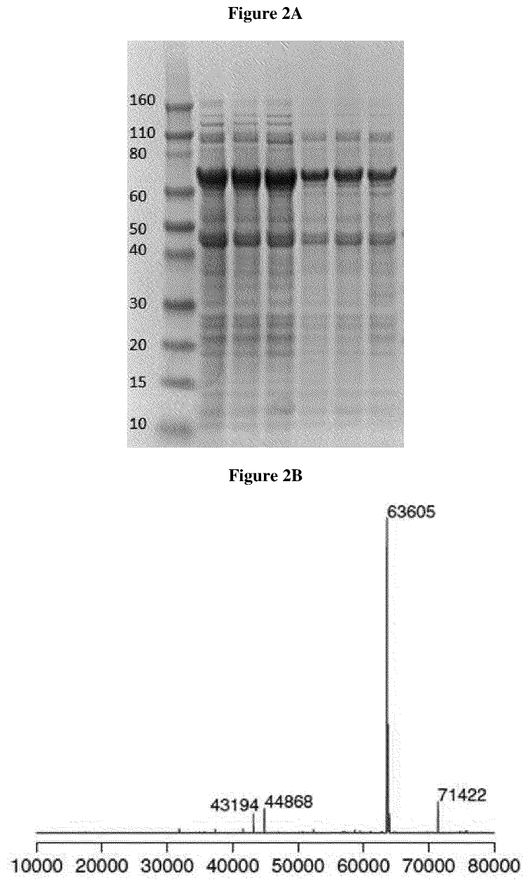

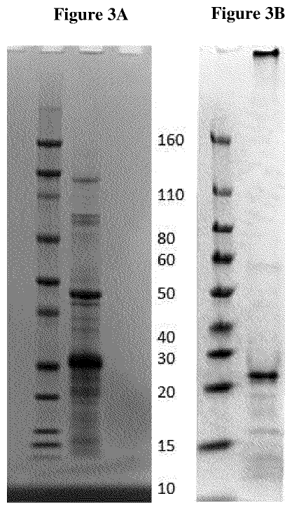

The purity and characterization of the 2Yx2A-MBP proteins by gel and LC/MS are shown in FIGS. 2A, 2B, 3A, 3B, 3C, and 3D. FIG. 2A shows a 4-12% Bis-Tris SDS PAGE Gel analysis of NiNTA purified 2Yx2A-MBP proteins under different IPTG induction conditions and either 20 or 6 hour time points. FIG. 2B shows a LC-MS (ESI-TOF) analysis of NiNTA purified construct with most stringent expression conditions: 6 h 0.1 mM IPTG. FIG. 3A shows a 4-12% Bis-Tris SDS PAGE Gel analysis of ion exchange purified 2Yx2A (75% pure by gel densitometry analyzed in ImageJ). FIG. 3B shows a 4-12% Bis-Tris SDS PAGE Gel analysis of Biotage HPLC purified 2Yx2A shows a single band corresponding to 2Yx2A monomer while also a large band that does not travel down the gel corresponding to the assembled protein that was not disassembled on the PAGE gel (>95% pure by densitometry analyzed in ImageJ). In both gels 2Yx2A complex runs at a higher apparent molecular weight, a phenomenon also observed with the IDP construct, which is likely due to its disordered nature. FIG. 3C shows a purification of 2Yx2A from MBP on poroshell column. 2Yx2A elutes at 8.2 minutes while MBP elutes at 10 min. A small amount of 2Yx2A also elutes around 9 minutes likely due to interactions with MBP. Only pure fractions are collected, for higher throughput purification, a C18 Biotage SNAP Bio 300A is used on a Biotage HPLC setup. FIG. 3D shows an LC-MS (ESI-TOF) analysis of ion exchange purified and HPLC purified 2Yx2A. The expected molecular weight of monomer: 18290.69. For the ion exchange purified protein 70% exists as a monomer (18291 Da), 10% as a dimer (36580 Da), and 19% impurity by MBP (45332 Da). For the Biotage HPLC purified protein, 76% exists as a monomer (cysteine residue capped by excess B-mercaptoethanol in the buffer at +76: 18367 Da), 23% as a dimer (36580 Da), and 0% impurity by MBP (45332 Da).

Expression and Purification of 2Yx3A-MBP. (f) Expression: 2Yx3A-MBP was expressed in the same way as 2Yx2A-MBP (b).

(g) Purification: 2Yx3A-MBP was purified in the same was as 2Yx2A-MBP (f).

In contrast to the expression of IDP-MBP and 2Yx2A-MBP, 2Yx3A-MBP resulted in low pellet volume during expressing. Additionally, after cell lysis by sonication, the protein supernatant resembled sudsy soap making it difficult to handle and hard to purify. The surfactant like nature of 2Yx3A-MBP was still present after NiNTA purification something not seen with the 2Yx2A-MBP construct. This is interesting because the only difference in the two sequences is the addition of four amino acids (YAYI) to the 2Yx3A-MBP sequence.

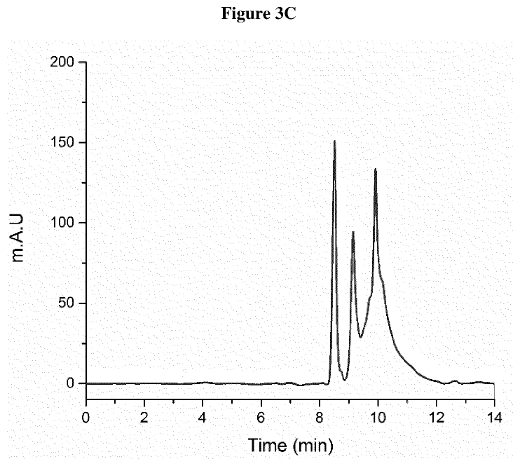

The characterization of the 2Yx3A-MBP proteins are shown in FIGS. 4A, 4B, and 4C. FIG. 4A is a photograph showing that after cell lysis, sonication, and filtration, the 2Yx3A-MBP crude protein mixture is very soapy. FIG. 4B is a photograph showing that after NiNTA purification of 2Yx3A-MBP construct, the protein mixture is still very soapy. FIG. 4C top graph: LC-MS (ESI-TOF) analysis of NiNTA purified 2Yx3A-MBP shows an impure mixture containing truncations of the 2Yx3A-MBP protein where the desired construct is obtained at 88% purity. FIG. 4C middle graph: LC-MS (ESI-TOF) analysis of 2Yx3A+MBP directly after cleavage by thrombin. The observed molecular weights indicate that this construct has a high propensity to assemble even in the presence of solubilizing MBP, staying in contact even during LC-MS TOF analysis. FIG. 4C bottom graph: Ion exchange purified 2Yx3A, showing that the purification of the construct from MBP was difficult, which is likely due to the ability of this construct to assemble even in the presence of MBP.

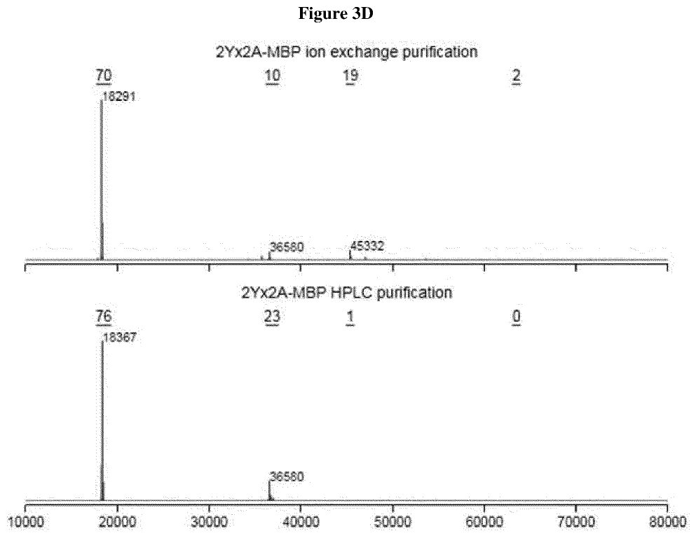

FIG. 5A is a schematic representation of the protein constructs described in this Example.

### Example 2: Micelle Characterization

This example provides characterization data of the micelles prepared from the exemplary fusion proteins described herein.

The resulting micelle from the 2Yx2A construct was characterized fully and in some cases the non-assembling IDP construct was used for comparison. The micelle from the 2Yx3A construct was not further characterized due to difficulties with expression and purification of the 2Yx3A construct. Briefly, to analyze the 2Yx2A, the lyophilized protein was resuspended in water and then adjusted to the desired buffer conditions.

Dynamic Light Scattering. DLS analysis was conducted on a Malvern Instruments Zetasizer Nano ZS. Data plots and standard deviations are calculated from an average of three measurements, each of which consisted of 13 runs. Measurement data is presented as a diameter determined by the % Number distribution. When measuring any of the assembling constructs, the protein module could be used to analyze the particles, however, to obtain any signal for IDP it must be treated as a polymer and diluted to low concentrations.

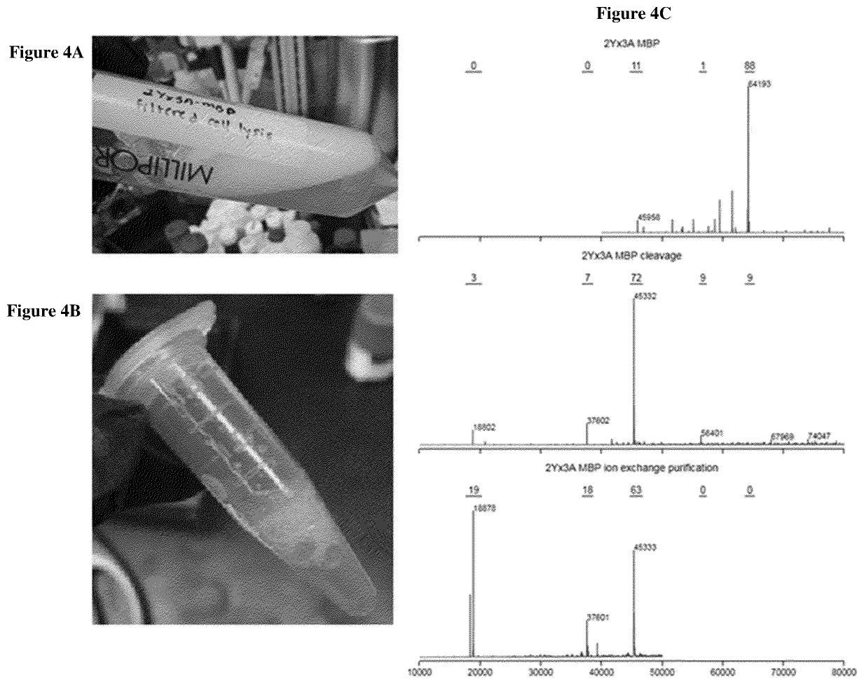

FIG. 6 is a chart showing DLS measurements of IDP (2 μM in Phosphate Buffer pH 5.3) and 2Yx2A construct (40 μM in 100 mM Phosphate buffer pH 5.3).

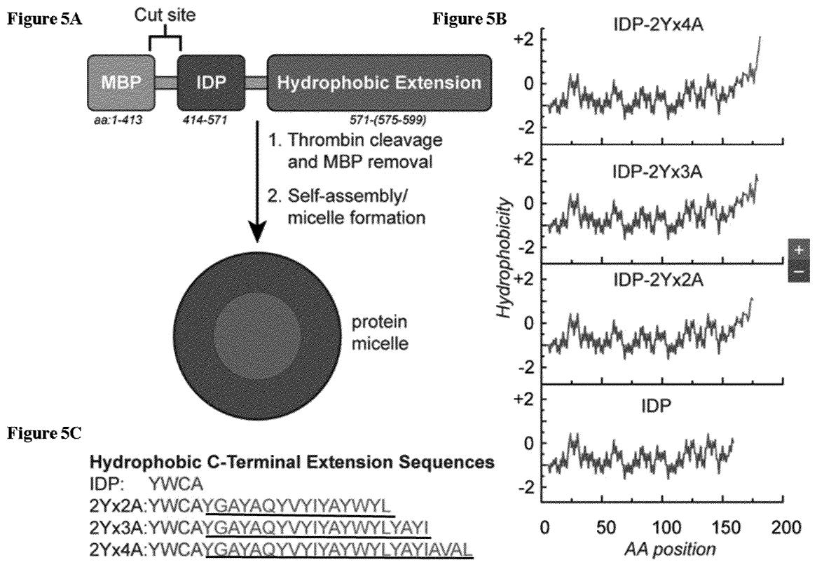

FIGS. 7A and 7B are charts showing the pH stability of the 2Yx2A construct as determined by DLS.

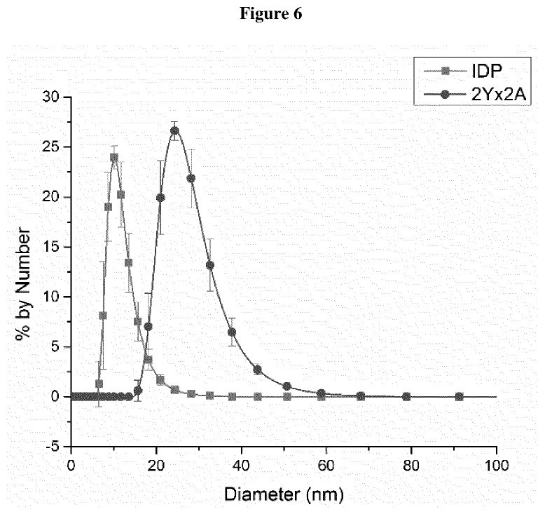

FIG. 8 is a chart showing the dependence of 2Yx2A micelle size on the concentration in 1×PBS pH 7.4 and 100 mM PB pH 5.3, where the trends closely reflect that of the CMC determined by the pyrene fluoresence assay.

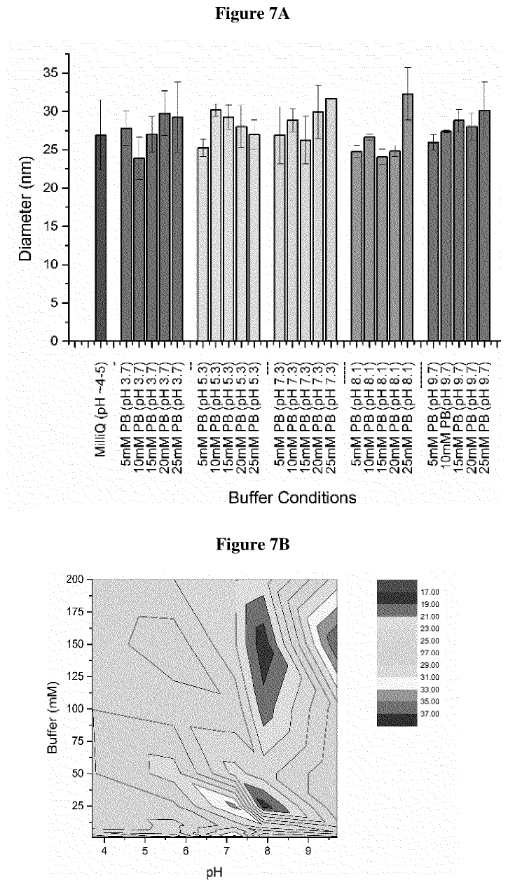

FIGS. 9A and 9B are charts showing the effects of temperature on the diameter of 2Yx2A micelles as determined by DLS.

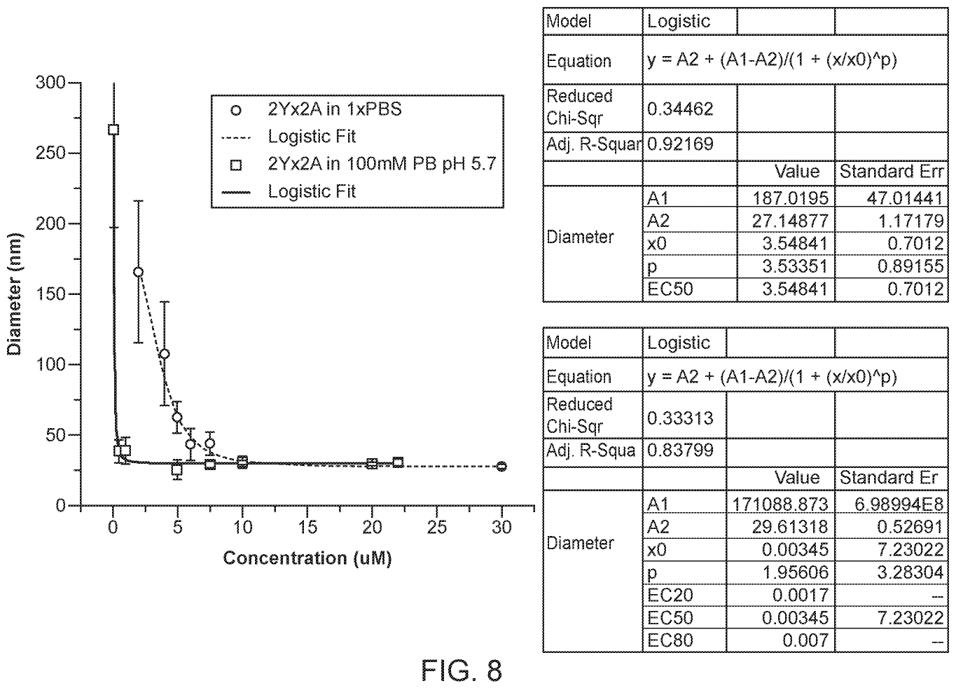

Size Exclusion Chromatography. FIG. 10 is a chart showing size exclusion chromatography LS9 traces of virus-like particle MS2 (known diameter 27 nm), IDP, and 2Yx2A micelles. The major peak for the 2Yx2A micelles overlaps that of MS2, further supporting the diameter reported from DLS measurements of 27.73 nm. IDP which shows a diameter of 11.25 nm on the DLS also elutes late indicating a smaller size.

Critical Micelle Concentration Determination by Pyrene Fluorescence. The CMC of 2Yx2A and IDP in 100 mM PB pH 5.8 was analyzed by measuring the first and third vibronic band of pyrene (I1/I3-ratio) which increases with increasing polarity of the probe environment. For example, the I1/I3 ratio in water is 1.32 while in cyclohexane is it 0.6.

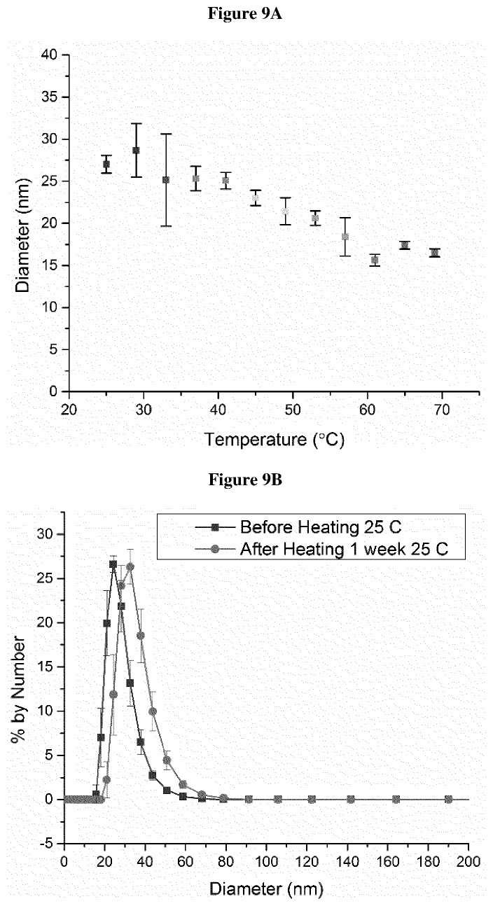

To each sample, 2 μM of pyrene was added and let equilibrate for 5 minutes. Each protein solution was then diluted with a solution of 2 μM pyrene in the buffer to keep pyrene and salt concentration constant, but decrease protein concentration. The emission spectrum of pyrene was collected on a Horiba fluorimeter exciting at 335 nm with a 5 nm window and monitoring emission from 350-800 nm. At higher protein concentrations a peak at 383 nm corresponding to pyrenes third vibronic band is present, while at lower concentrations the intensity of this band decreases (FIGS. 11A and 11B).

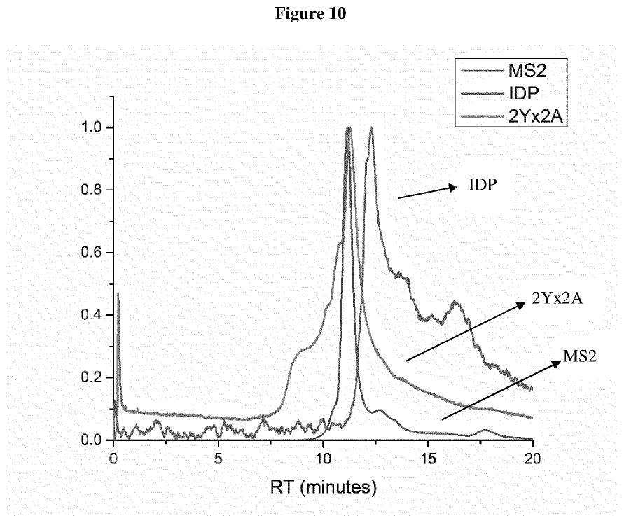

When the I1/I3 ratio is plotted against 2Yx2A protein concentration, a Boltzmann fit can be applied to the data points to calculate an EC₅₀ value. In 1×PBS pH 7.4 this value is 26 μM while in 100 mM PB pH 5.7 this value is 13 μM. Alternatively, when this same analysis is applied to IDP, the I1/I3 ratio remains constant and exhibits that of water (0 uM protein) overall concentrations up to 100 μM. The I1/I3 ratio of pyrene in high concentrations (above 30 μM) of 2Yx2A closely resembles that of pyrene in 1-propanol ˜1.0 (FIG. 12).

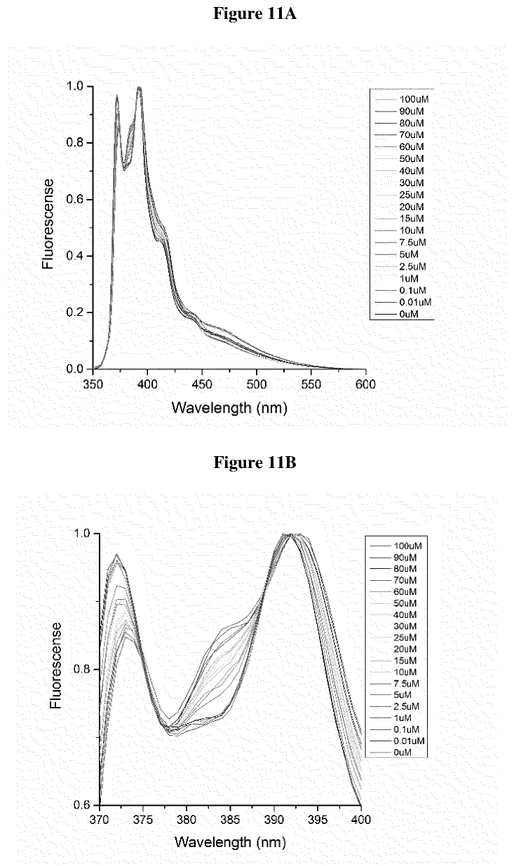

Micelle exchange dynamics measured using FRET. (a) Labeling of internal cysteine with fluorescent dye: To measure the exchange dynamics of the 2Yx2A micelles, two separate populations of 2Yx2A were labeled with either Fluorescein maleimide or Rhodamine red maleimide on the internal cysteine residues that sits at between the hydrophobic and hydrophilic portion of the protein. After 24 h, both populations showed 4% labeling (FIG. 13).

To remove excess dye, the proteins were purified using a NAPS column resulting in 600 μL of protein at 1 μM (below the CMC). To this solution, 50 μL of 40 uM 2Yx2A was added and then spin concentrated with a 3 KDa MWCO. This should achieve approximately 1% labeling of all 2Yx2A protein monomers. Assuming aggregation numbers in the hundreds this correlates to a low average number of dye molecules per micelle.

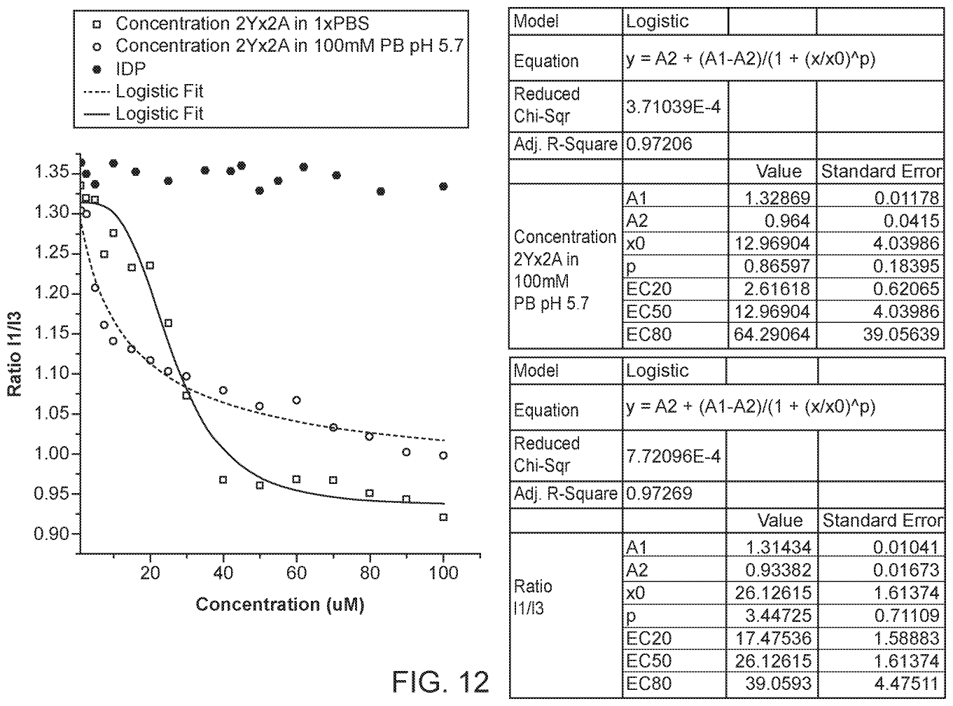

(b) FRET analysis: 2Yx2A-FITC and 2Yx2A-RhoRED were individually excited using a Horiba fluorometer with 490 nm light 5 nm window and the emission spectrum was collected from 500-800 nm in 1 nm increments. Then 30 μL of each solution was mixed together and then immediately analyzed. Consecutive time points over 40 hs were then taken. FIG. 14A is a FRET analysis of 2Yx2A when excited with 490 nm light the emission of fluorescein is observed at 515 nm whereas the emission of rhodamine is observed at 580 nm. FIG. 14B is a chart demonstrating that the FRET ratio, defined at I580/(I580+I515) can be plotted against time and fit to a logarithmic equation. By 75 minutes, 50% mixing of the micelles is achieved, indicating that our micelles are dynamic in nature.

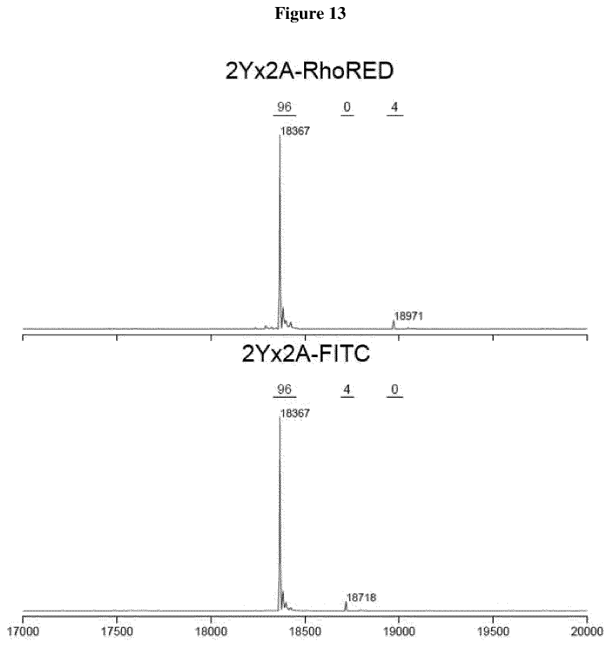

Cryo TEM. Cryo TEM samples were prepared from a 12 μM stock solution of 2Yx2A in 100 mM PB pH 5.3 before analysis samples were diluted 30-fold to a concentration of 0.4 μM for analysis. By DLS the average diameter and standard deviation observed for these particles was slightly larger due to the dilution (see FIG. 8) reporting an average diameter of 48.43±10.62 nm. Images were exposed deiced by pre-exposing the grid to photons prior to image acquisition. Embedded in the vitrified ice, spherical micelles are observed. Image analysis using ImageJ reveals an average diameter of 50.46±12.14 nm which closely that of the DLS results (FIG. 15). Additionally, in some particles, a core-shell like structure can be observed.

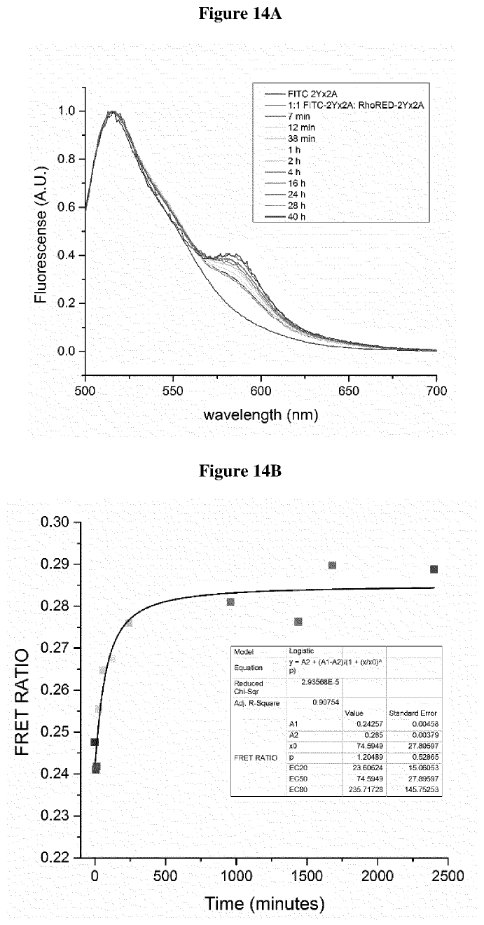

FIG. 16 is a chart showing core-shell diameters of 10 micelles. The corresponding measurements for the shell diameter, core diameters, and shell thickness are shown in the below Table 7. With increasing core size, there is an increase in shell size. The thickness, defined as the distance between an individual micelles core and shell, for these micelles was on average 12.23±3.95 nm, which is close to the expected length of the intrinsically disordered hydrophilic region of the construct.

TABLE 7

| Shell Diameter | Core diameter | Difference | Shell thickness |
|---|---|---|---|
| (nm) | (nm) | (nm) | (nm) |
| 51.08 | 31.16 | 19.92 | 9.96 |
| 57.60 | 32.85 | 24.75 | 12.37 |
| 50.72 | 30.05 | 20.67 | 10.33 |
| 49.82 | 34.55 | 15.27 | 7.63 |
| 44.73 | 27.98 | 16.74 | 8.37 |
| 78.05 | 41.35 | 36.69 | 18.34 |
| 55.00 | 33.69 | 21.30 | 10.65 |
| 71.53 | 34.75 | 36.78 | 18.39 |
| 54.56 | 34.02 | 20.54 | 10.27 |
| 64.93 | 33.04 | 31.88 | 15.94 |

SAXS. This work benefited use of the SasView application(M. Doucet, et al. SasView Version 4.1.2).

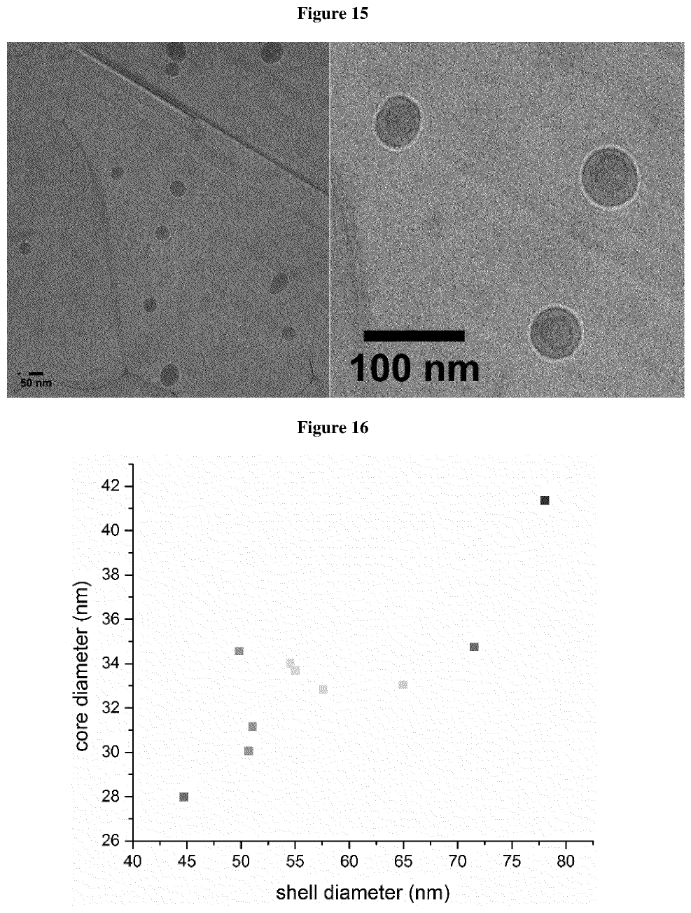

FIGS. 17A and 17B are charts showing the Rg and P(r) distribution of the 2Yx2A. FIG. 17A is a chart showing SAXS scattering curve of 68 and 34 μM 2Yx2A in 100 mM PB pH 5.7 and 32 μM 2Yx2A in 1×PBS. The fit of the curve is used to determine the real space Rg and the P(r) distribution. FIG. 17B is a chart showing results of the P(r) distribution fit. All three curves appear very similar resulting in real space Rg values that are all approximately 10 nm. The Rh obtained from DLS measurements is 13.08 nm resulting in an Rg/Rh ratio of 0.76, consistent with a packed spherical micelle. Additionally, the average radius can be determined for the three samples, where they all show maximum probability between 10 and 15 nm and going to zero probability (dmax) around 320 nm.

### Example 3: Loading 2Yx2A Micelles with Pyraclostrobin

This example is illustrative in demonstrating that the micelles prepared from the exemplary fusion proteins described herein are capable of being loaded with a hydrophobic compound, such as pyraclostrobin.

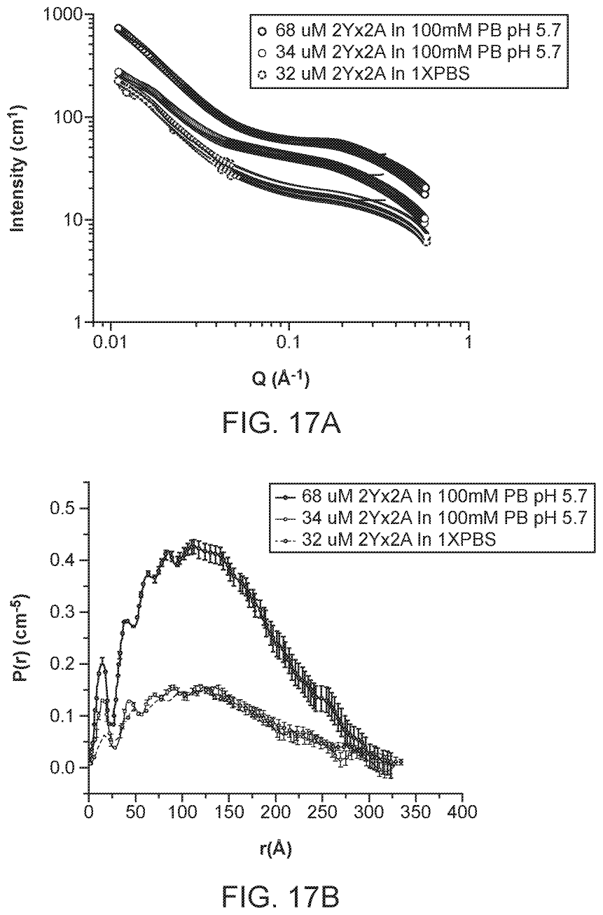

Pyraclostrobin is a highly water-insoluble organic compound that is also a potent fungicide. Its solubility in water is reported to be 1.9 mg/L. As a control a saturated solution of pyraclostrobin was created by adding it to 100 mM PB pH 5.3 and measuring its absorbance at 280 nm, as expected there was no observable signal. To determine if pyraclostrobin could be taken up into 2Yx2A micelles, pyraclostrobin was added until it saturated a solution of 100 μL of 11 μM (A280: 0.361) 2Yx2A, evidenced by yellow particulates. The solution was then centrifuged and the supernatant was removed and then placed in a fresh tube and centrifuged again. Supernatant removal and centrifugation were repeated 3 times to remove any insoluble pyraclostrobin that has not partitioned into the micelle interior. The solution was then measured with a nanodrop to determine its absorbance at 280 nm resulting in an A280 of 0.502 (+0.141 mAU). Given the pyrene extinction coefficient of 24,000 at A275, this increase in absorbance corresponds to the presence of 5.8 μM pyrene. Similarly, the same sample can be analyzed for pyraclostrobin content via HPLC using a calibration curved developed using known pyrene concentrations in acetonitrile (FIG. 18A). For HPLC analysis the protein-pyraclostrobin solution is diluted to ½ of its volume by pyrene to break apart the micelles. Then a known volume is injected onto the HPLC (FIG. 18B). Based on the area of the pyrene peak and volume injected the number of moles and thus the concentration of pyraclostrobin can be determined. Using this method, 7.37 μM pyraclostrobin is encapsulated in 11 μM of protein.

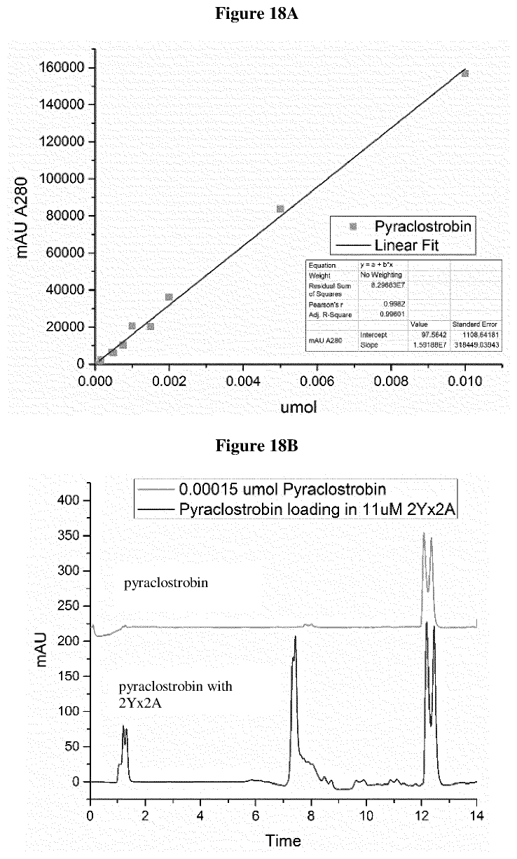

Instead of directly adding pyraclostrobin to a solution of 2Yx2A micelles an alternative method was used to achieve higher loading efficiency. This method involved the co-resuspension of lyophilized protein and pyraclostrobin in THF which is then slowly diluted with buffer. The solution was then centrifuged and the supernatant was removed and then placed in a fresh tube and centrifuged again. Supernatant removal and centrifugation were repeated 3 times to remove any insoluble pyraclostrobin that has not partitioned into the micelle interior. The sample was then lyophilized to remove all solvent and then resuspended in milliQ water. Although no visible precipitation was observed, the sample was centrifuged to remove any unincorporated pyraclostrobin. For HPLC analysis the protein-pyraclostrobin solution is diluted to ½ of its volume by pyrene to break apart the micelles. Then a known volume is injected onto the HPLC. Based on the area of the pyrene peak and volume injected the number of moles and thus the concentration of pyraclostrobin can be determined. FIG. 19 shows that the average mole ratio of Pyraclostrobin: 2Yx2A protein monomers was determined to be 15.2±8:1.

Accordingly, these results demonstrate that the micelles of the present technology are useful in compositions for solubilizing highly water-insoluble organic compounds and can be useful in methods for delivery of such compounds to an intended target. For example, micellar compositions comprising pyraclostrobin may be useful as a fungicide.

### Example 4: TEM of Pd(dppf)Cl₂ Loaded 2Yx2A Micelles

This example is illustrative in demonstrating that the micelles prepared from the exemplary fusion proteins described herein are capable of being loaded with a hydrophobic metal complex.

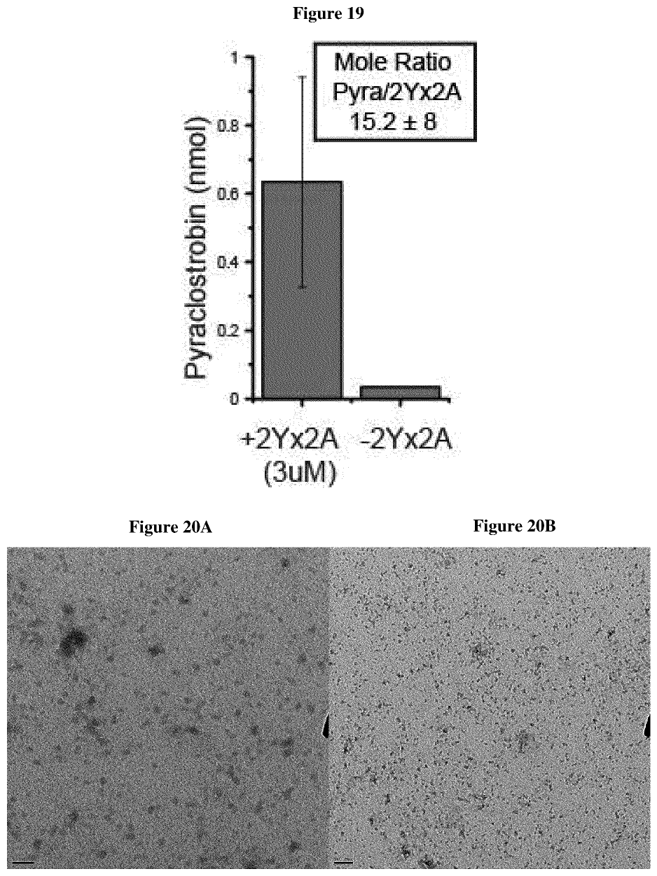

To visualize the ability of the 2Yx2A micelles to load hydrophobic compounds in their core, 40 μM 2Yx2A protein was incubated with the commonly used Suzuki coupling catalyst Pd(dppf)Cl₂ for 1 week at 4° C. Prior to use, the sample was centrifuged and then the supernatant was passed through a 2 μm spin filter to remove any insoluble catalyst that has not partitioned into the micelle interior. The sample was then placed onto formvar-coated carbon grids that had been hydrophilized and let sit for 2 minutes. Excess liquid was wicked away using the tip of filter paper. The grids were then dried completely prior to analysis by a TECANI 12 TEM (UC Berkeley Electron Microscopy Facility). Due to the high electrons density of the palladium and iron-containing ferrocene ligands, the contrast in the TEM images can be achieved without the use of stain (FIGS. 20A and 20B).

### Example 5: Preparation of Fusion Proteins

This example describes another method for preparing the exemplary fusion proteins described herein.

This example describes an expression system where the solubilizing fusion protein, maltose binding protein (MBP), has been replaced with the inclusion body directing fusion protein ketosteroid isomerase (KSI). By installing KSI at the N-terminus of the IDP-2Yx2A protein sequence, the entire fusion protein was sent to insoluble inclusion bodies during expression. Due to the insoluble nature of the fusion protein, the fusion protein was easily purified by centrifugation after cell lysis. Use of the KSI moiety reduced the amount of solvent needed in the initial purification step and also avoided stability issues encountered with soluble proteins. To remove the KSI fusion protein from the IDP-2Yx2A sequence, a methionine (Met) residue was installed between the two protein domains (KSI-Met-IDP-2Yx2A). Upon exposure to cyanogen bromide (CNBr), the peptide bond at the C-terminus of the Met residue was hydrolyzed leaving a C-terminal lactone on the KSI fusion protein and the desired IDP-2Yx2A. The IDP-2Yx2A protein was then purified using reversed phase chromatography.

The following materials were used:
1000× Carbenicillin solution(Carb) was prepared by dissolving 100 mg/mL of solid antibiotic in MilliQ H₂O and was stored at −20° C. prior to use.
1000× Chloramphenicol solution(Cam) was prepared by dissolving 25 mg/mL in EtOH and was stored at −20° C. prior to use.
LB agar plates+Carb was prepared by combining 12.5 g of LB broth (powder) from Thermo Fisher Scientific and 7.5 g agar in 500 mL of MilliQ water. The solution was autoclaved and allowed to cool to 55° C. 500 uL of 1000× (100 mg/mL) stock of Ampicillin was added by swirling to mix and resulted in a final concentration of 100 ug/mL. The workbench was sterilized by using 70% EtOH and by turning on the Bunsen burner. 30-40 mL was poured into each 10 cm petri dish. The plates were allowed to dry for 5 min with lid half way on and then the lids were closed. Once the agar solidified, the plates were stacked into columns and allowed to dry for an additional 3 h at ambient temperature. The plates were stored at 4° C. prior to use.
LB Agar plates with Carb and Cam were prepared as follows. LB agar plates with Carb were removed from 4° C. fridge one hour prior to use and allowed to warm at 37° C. for 30 minutes. Under sterile conditions, 30 μL of the 1000× Cam solution was spread onto the plates. The plates were returned to the 37° C. incubator for 30 mins and were then ready for use.
TB media was prepared from 8 mL glycerol 47.5 g terrific broth powder (Sigma Aldrich) and 1000 mL MilliQ H₂O.
LB media was prepared from 25 g of Luria broth powder (Sigma Aldrich) and 1000 mL MilliQ H₂O.
Lysis buffer was prepared from 20 mM Hepes, 300 mM NaCl, 10 mM Bme, 0.1% Triton X, and MilliQ H₂O.

Pet31b-KSI-IDP-2Yx2A Plasmid Construction

Entry vector: The pet31b KSI entry vector was purchased from Millipore as 69952 Sigma-AldrichpET-31b(+) DNA—Novagen.
Template DNA: The MBP-IDP-2Yx2A pet28b vector was used as template DNA and overhang PCR was performed to amplify the IDP-2Yx2A sequence.
Overhang PCR primers: These were purchased from Integrated DNA Technologies.

Forward Primer: F2 xho1 alwnl pet 31b
(SEQ ID NO: 62)
5′-AAC TAT AAT ATA TAC AGA TGC TGG CAG AGG
CCA AGA GT-3′;
MW = 11,767.7 g/mol.
Reverse Primer: R2 xho1 alwnl pet 31b
(SEQ ID NO: 63)
5′-AAA TTC CCA AAA CTC GAG CAT CAG ATA CCA
ATA CGC ATA AA-3′;
MW = 12,516.2 g/mol.

As shown below in Table 8, overhang PCR was performed on a BioRad S1000 Thermal Cycler with an annealing temperature of 55° C. Following heat inactivation of the Phusion enzyme, 6 μL of 6× loading dye was added to each overhang PCR reaction, which was then loaded onto a 1% agarose gel pre-stained with SYBR Safe (ThermoFisher). Gel electrophoresis was performed at 120 V for 35 minutes and then imaged under UV fluorescence with a BioRad GelDoc EZ Imager. The DNA bands corresponding to approximately 500 base pairs were excised from the gel and both were placed in a 1.5 mL Eppendorf tube. The amplified PCR product was removed from the gel using the Quick Gel Extraction Kit (Promega). The amplified PCR product was eluted in a final volume of 50 μL with a concentration of 23.7 ng/μL.

TABLE 8
Overhang PCR of IDP-2Yx2A

| | PCR 1 (μL) | PCR 2 (μL) |
|---|---|---|
| Template DNA (MBP-IDP-2Yx2A pet28b | 0.5 | 0.5 |
| vector) | | |
| Reverse primer (10 μM) | 1 | 1 |
| Forward primer (10 μM) | 1 | 1 |
| 5X Phusion buffer | 4 | 4 |
| dNTP | 0.4 | 0.4 |
| DMSO | — | 0.6 |
| MilliQ H₂O | 12.6 | 12 |
| *Phusion | 0.5 | 0.5 |
| Total volume | 20 | 20 |

Table 7. *Phusion DNA Pol was added last

The amplified PCR product and the pet31b entry vector were then digested with XhoI and AlwnI restriction enzymes to create complementary sticky ends. The parameters for the restriction digest are shown in Table 9.

TABLE 9
Restriction digest of pet31b entry
vector and amplified PCR product

| | Insert | Vector |
|---|---|---|
| MilliQ H₂O | — | 49 |
| 1 μL Xhol | 1 | 1 |
| 1 μL AlwnI | 1 | 1 |
| CutSmart Buffer (NEB) | 6 | 6 |
| Amplified PCR product (23.7 ng/μL) | 50 | — |
| Pet31b entry vector | — | 1 |
| Total volume | 58 | 58 |

The restriction digests were incubated for 1 h at 37° C. and then heat inactivated at 80° C. for 20 min. Following digestion, 10 μL of 6× loading dye was added to each sample, which was then loaded onto a 1× agarose gel pre-stained with SYBR Safe (ThermoFisher). Gel electrophoresis was performed at 120 V for 35 minutes and then imaged under UV fluorescence with BioRad GelDoc EZ Imager. Fluorescent bands corresponding to approximately 500 base pairs (insert) and 5000 base pairs (vector) were excised from the gel and purified using the quick gel extraction kit (Promega). After elution, the concentrations of the cut vector and insert DNA was 15.1 ng/μL and 13.9 ng/μL respectively.

The ligation reaction between the digested pet31b (vector) and digested amplified PCR product (insert) was performed with varying ratios of vector: insert, where the vector concentration was kept constant at 5 ng/μL. The parameters for the ligation of the digested pet31B vector is shown in Table 10.

TABLE 10
Ligation of digested pet31B vector and amplified PCR product

| Vector DNA:Insert DNA | 1:0 | 1:1 | 1:3 | 1:7 |
|---|---|---|---|---|
| T4 DNA ligase buffer | 2 μL | 2 μL | 2 μL | 2 μL |
| Vector DNA (15.1 ng/μL) | 7 μL | 7 μL | 7 μL | 7 μL |
| Insert DNA (13.9 ng/μL) | — | 1 μL | 2.5 μL | 6.5 μL |
| Nuclease free water | 10 μL | 9 μL | 7.5 μL | 3.5 μL |
| T4 DNA ligase | 1 μL | 1 μL | 1 μL | 1 μL |
| Total volume | 20 μL | 20 μL | 20 μL | 20 μL |

The ligation reactions were incubated at 16° C. for 16 h and heat inactivated at 80° C. for 20 min. Half of each ligation reaction (10 μL) was transferred to separate 1.5 mL Eppendorf tubes containing 50 μL of frozen XL1 blue chemically competent cells. The ligation reactions were gently flicked to ensure proper mixing and then incubated on ice for 30 min. The transformation was performed by heat shocking the cells-ligation mixture in a 42° C. water bath for 42 secs and then put back on ice. Under sterile conditions, 950 μL of SOC media was immediately added to the Eppendorf tube, which was then placed in a 37° C. incubator with an orbital rotator set to 200 rpm. After 1 h, 200 μL of each of the four transformations were plated on separate carbenicillin agar plates, labeled with their respective insert: vector ratios, and placed in a 37° C. incubator overnight.

After overnight incubation, the transformations corresponding to the 1:3 and 1:7 ligations had the most colonies with the 1:0 negative control had fewer than 5 colonies suggesting a low background of the vector with unincorporated insert. Four colonies from each plate were scraped with a sterile pipette tip and transferred to 5 mL of LB media+carbenicillin and grow overnight in a 37° C. incubator with an orbital rotator set to 200 rpm. The overnight cultures were transferred to 1.5 mL Eppendorf tubes and centrifuged at 13.1 g for 2 min to pellet the cells. The supernatant was removed and the plasmids were extracted from the pelleted cells using a QIAGEN miniprep kit. The eluted plasmids were sent for sequencing using a T7 forward primer. Of the eight plasmids sent for sequencing, all contained the IDP-2Yx2A insert at the C-terminus of the KSI fusion protein as shown below.

KSI-IDP-2Yx2A:
(SEQ ID NO: 61)
MHTPEHITAVVQRFVAALNAGDLDGIVALFADDATVEDPVGSEPRSG
TAAIREFYANSLKLPLAVELTQEVRAVANEAAFAFTVSFEYQGRKTV
VAPIDHFRFNGAGKVVSIRALFGEKNIHACQMLAEAKSPAEAKSPAE
VKSPAVAKSPAEVKSPAEVKSPAEAKSPAEAKSPAEVKSPATVKSPG
EAKSPAEAKSPAEVKSPVEAKSPAEAKSPASVKSPGEAKSPAEAKSP
AEVKSPATVKSPVEAKSPAEVKSPVTVKSPAEAKSPVEVKSPYWSAY
GAYAQYVYIYAYWYLM [symbol] ;
UPPER CASE = KSI polypeptide
UNDERLINE UPPER CASE = CNBr cleavage site
BOLD UPPER CASE = IDP polypeptide
UNDERLINED BOLD UPPER CASE = Hydrophobic
polypeptide [symbol]
= His-tag

Expression of KSI-IDP-2Yx2A. The pet31b plasmid containing KSI-IDP-2Yx2A was transformed into Rosetta2plys cells for expression by adding 1 μL of the plasmid to 50 μL Rosetta2plys cells in a 1.5 mL Eppendorf tube on ice. The cells were then gently flicked to ensure proper mixing and incubated on ice for 30 mins before heat shocking the cells in a 42° C. water bath for 42 secs. Under sterile conditions, 950 μL of SOC media was immediately added to the Eppendorf tube containing transformation, which was then placed in a 37° C. incubator with an orbital rotator set to 200 rpm. After 1 h, 200 μL of the transformation was plated on carbenicillin+chloramphenicol agar plates and placed in a 37° C. incubator overnight.

After overnight incubation, a single colony from the agar plate was picked using a sterile pipette and added to 15 mL of LB media with carbenicillin+chloramphenicol and incubated overnight. The following day the entire overnight culture was added to 1 L of sterile TB media with carbenicillin+chloramphenicol in a 4 L flask. The flask was then placed in a 37° C. incubator rotating at 200 rpm. When the optical density at 600 nm reached 0.7 the culture was cooled down to 18° C. for 30 minutes. To induce expression, 0.5 mM IPTG was added to the media and the culture was shaken at 200 rpm for an additional 18 h overnight.

After overnight expression, the culture was centrifuged at 4000 rpm for 15 minutes. The supernatant discarded, and the remaining cell pellet was split evenly and transferred to two 50 mL falcon tubes resulting in a total cell pellet weight of 10 g (5 g per falcon tube). The cell pellets were then frozen at −20° C. overnight.

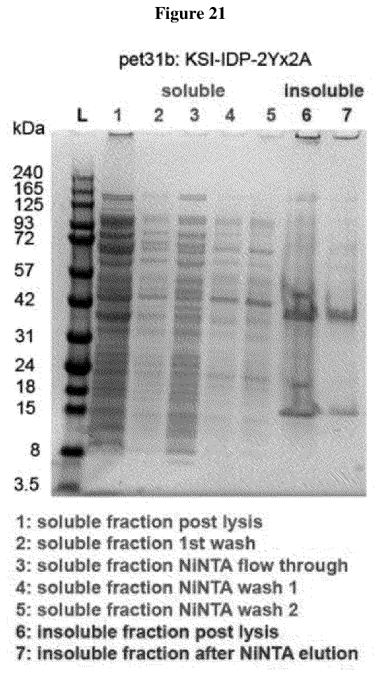

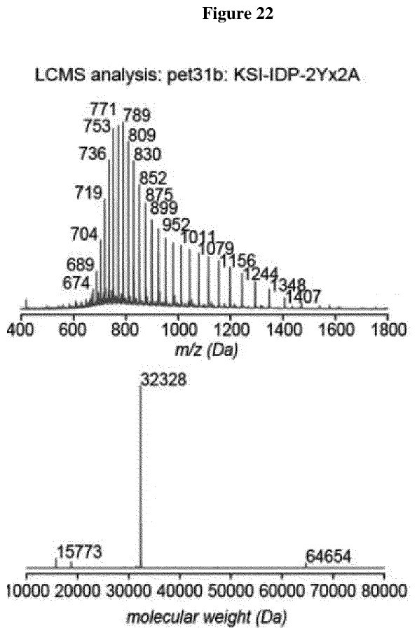

Purification of KSI-IDP-2Yx2A. The frozen 5 g cell pellet was resuspended in 30 mL of lysis buffer (20 mM HEPES, 300 mM NaCl, 10 mM BMe, 0.1% Triton-X) plus 300 μL PMSF immediately before use. The cells were lysed by sonication on the ice at 70% amplitude for 30 minutes (2 secs on 4 secs off). The lysed cells were then centrifuged at 14000 rpm for 20 minutes. The supernatant was removed and discarded. The pellets were then resuspended in lysis buffer and centrifuged at 14000 rpm again. The supernatant was discarded and the pellets were then resuspended and centrifuged two more times in MilliQ H₂O. The resulting pellet was then resuspended in 10 mL of 6M Guanidinium HCl and centrifuged at 14000 rpm. The desired KSI-IDP-2Yx2A protein now resided in the supernatant at which is removed from the pelleted cell debris and stored at 4 C. The protein concentration obtained in the insoluble fraction from one 5 g cell pellet (500 mL of cell culture) is approximately 180 mg by UV absorbance at 280 nm, where the approximation that an A280 of 1.0=1 mg/mL. Of the 180 mg of protein obtained, the centrifugation purification process produced a protein solution which was predominately KSI-IDP-2Yx2A when analyzed by SDS PAGE gel and LCMS (FIGS. 21 and 22, respectively).

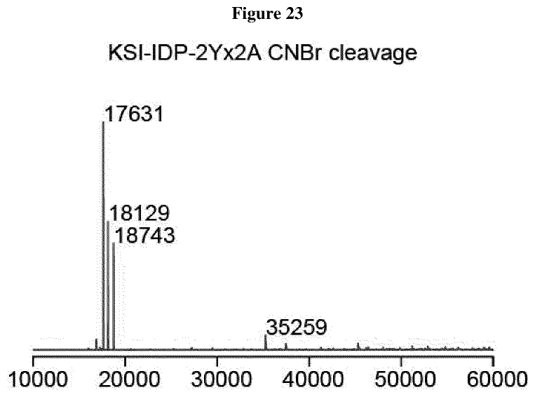

CNBr cleavage of KSI-IDP-2Yx2A. To the 10 mL solution of KSI-IDP-2Yx2A in 6M Guanidinium HCl, 2 mL of 3M HCl and one scoop (˜50 mg) of cyanogen bromide (CNBr) was added. The solution container was wrapped in foil and stirred under nitrogen overnight. The following day complete cleavage was observed by LCMS as no mass corresponding to of the original KSI-IDP-2Yx2A was detected and the mass of IDP-2Yx2A was also observed (FIG. 23).

HPLC Purification of IDP-2Yx2A. The IDP-2Yx2A was then then purified using reversed phase chromatography as previously described herein.

### EQUIVALENTS

While certain embodiments have been illustrated and described, a person with ordinary skill in the art, after reading the foregoing specification, can effect changes, substitutions of equivalents and other types of alterations to the protein fusions and micelles of the present technology or derivatives, or pharmaceutical compositions thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the present technology. This includes the generic description of the present technology with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

### REFERENCES

1. Vargo, K. B., Parthasarathy, R. & Hammer, D. A. Self-assembly of tunable protein suprastructures from recombinant oleosin. Proc. Natl. Acad. Sci. U.S.A 109, 11657-62 (2012).
2. Petka, W. A., Harden, J. L., McGrath, K. P., Wirtz, D. & Tirrell, D. A. Reversible Hydrogels from Self-Assembling Artificial Proteins. Science (80-.). 281, 389 LP-392 (1998).
3. Park, W. M. & Champion, J. A. Thermally Triggered Self-Assembly of Folded Proteins into Vesicles. J. Am. Chem. Soc. 136, 17906-17909 (2014).
4. Huber, M. C. et al. Designer amphiphilic proteins as building blocks for the intracellular formation of organelle-like compartments. Nat. Mater. 14, 125-132 (2015).
5. Wen, Y. & Li, J. Ultrastable micelles boost chemotherapy. Nat. Biomed. Eng. 2, 273-274 (2018).
6. Fuguet, E., Ràfols, C., Rosés, M. & Bosch, E. Critical micelle concentration of surfactants in aqueous buffered and unbuffered systems. Anal. Chim. Acta 548, 95-100 (2005).
7. Adiga, S. P. & Brenner, D. W. Molecular Basis for Neurofilament Heavy Chain Side Arm Structure Modulation by Phosphorylation. J. Phys. Chem. C 114, 5410-5416 (2010).
8. Chang, R., Kwak, Y. & Gebremichael, Y. Structural Properties of Neurofilament Sidearms: Sequence-Based Modeling of Neurofilament Architecture. J. Mol. Biol. 391, 648-660 (2009).
9. Bhagawati, M. et al. Site-Specific Modulation of Charge Controls the Structure and Stimulus Responsiveness of Intrinsically Disordered Peptide Brushes. Langmuir 32, 5990-5996 (2016).
10. Raran-Kurussi, S. & Waugh, D. S. Unrelated solubility-enhancing fusion partners MBP and NusA utilize a similar mode of action. Biotechnol. Bioeng. 111, 2407-11 (2014).
11. Piñeiro, L., Novo, M. & Al-Soufi, W. Fluorescence emission of pyrene in surfactant solutions. Adv. Colloid Interface Sci. 215, 1-12 (2015).
12. Guler, M. O., Claussen, R. C. & Stupp, S. I. Encapsulation of pyrene within self-assembled peptide amphiphile nanofibers. J. Mater. Chem. 15, 4507 (2005).

1. An amphiphilic fusion protein having a formula S/I—X—H₁—H₂, wherein S— is a solubilizing moiety, I— is an insolubilizing moiety, —X— is a peptide sequence comprising a proteolytic or chemical cleavage site, —H₁— is a hydrophilic peptide, and —H₂ is a hydrophobic peptide, and the —H₁— comprises an intrinsically disordered peptide (IDP) sequence.

2. (canceled)

3. The amphiphilic fusion protein of claim 1, wherein one or more of:
(a) the IDP sequence comprises one or more polypeptide sequences from a human neurofilament protein, a San1 protein, an Hsp-33 protein, an E1A protein, a PhD protein, a Sic1 protein, a WASP protein, a p27 protein, a CREB protein, a PUP protein, or a LEA protein;
(b) the IDP comprises a human neurofilament polypeptide sequence and the human neurofilament polypeptide sequence comprises the amino acid sequence as set forth in SEQ ID NO: 2 or SEQ ID NO: 69;
(c) the IDP comprises repeats of the sequence (SPAEAK)ₙ (SEQ ID NO: 3) or repeats of the sequence (SPAEAR)ₙ (SEQ ID NO: 4), where n is an integer from 2 to 50, and/or
(d) the IDP comprises repeats of the sequence (SPAX₁AX₂)ₙ (SEQ ID NO: 53), where X₁ and X₂ are each any charged amino acid and n is an integer from 2 to 50.

4-7. (canceled)

8. The amphiphilic fusion protein of claim 1, wherein:
(a) the —H₂ comprises a hydrophobic polypeptide sequence comprising a tyrosine-rich amino acid sequence 5-20 residues in length; or
(b) the —H₂ comprises a hydrophobic polypeptide sequence selected from the group consisting of: YGAYAQYVYIYAYWYL (SEQ ID NO: 5), YGAYAQYVYIYAYWYLYAYI (SEQ ID NO: 6), YGAYAQYVYIYAYWYLYAYIAVAL (SEQ ID NO: 54), WEAKLAKALAKALAKHLAKALAKALKACEA (SEQ ID NO: 7), YWCCA(X)ₐ (SEQ ID NO: 8) where a is a number of any hydrophobic residue (X), YWXXV_{b}A_{b} (SEQ ID NO: 9) where b is an integer of 3 or greater and X is any hydrophobic residue, and YWA(X)_{c} (SEQ ID NO: 10) where c is a number of any hydrophobic residue (X).

9. (canceled)

10. The amphiphilic fusion protein of claim 1, wherein one or more of:
(a) the S— comprises one or more of a maltose binding protein (MBP) polypeptide sequence, a small ubiquitin-like modifier (SUMO) polypeptide sequence, a glutathione S-transferase (GST) polypeptide sequence, a SlyD polypeptide sequence, a NusA polypeptide sequence, a thioredoxin polypeptide sequence, a ubiquitin polypeptide sequence, or a T7 gene 10 polypeptide sequence;
(b) the S— further comprises a polyhistidine tag (His-tag);
(c) the S— comprises a MBP polypeptide sequence and comprises an amino acid sequence set forth in SEQ ID NO: 12;
(d) the —X— comprises a proteolytic cleavage site selected from a thrombin cleavage site, a tobacco etch virus (TEV) cleavage site, a 3C cleavage site, an enterokinase cleavage site, or a Factor Xa cleavage site; or
(e) the —X— comprises a thrombin cleavage site comprising the polypeptide sequence LVPR (SEQ ID NO: 13).

11-15. (canceled)

16. The amphiphilic fusion protein of claim 1, wherein one or more of:
(a) the I— comprises a ketosteroid isomerase polypeptide sequence;
(b) the I— comprises an amino acid sequence set forth in SEQ ID NO: 55; or
(c) the —X— comprises a chemical cleavage site selected from a CNBr cleavage site that cleaves at a methionine residue or a 2-nitro-5-thiocyanobenzoic acid cleavage site that cleaves at a cysteine residue.

17-18. (canceled)

19. The amphiphilic fusion protein of claim 1, wherein the fusion protein further comprises a cell targeting peptide (-T-) between the —X— and the —H₁—, such that the amphiphilic fusion protein has the formula S/I—X-T-H₁—H₂.

20. The amphiphilic fusion protein of claim 19, wherein the -T- is selected from the group consisting of a chitin binding domain (CBD), a cancer cell-targeting peptide, and an antimicrobial peptide.

21. The amphiphilic fusion protein of claim 20, wherein the -T- is a cancer cell-targeting peptide selected from the group consisting of a peptide targeting human head and neck solid tumors and having the amino acid sequence TSPLNIHNGQKL (SEQ ID NO: 18), a peptide targeting tumor neovasculature and having the amino acid sequence CGKRK (SEQ ID NO: 19), a peptide targeting breast carcinoma and having the amino acid sequence CGNKRTRGC (SEQ ID NO: 20), a peptide targeting prostate vasculature and having the amino acid sequence SMSIARL (SEQ ID NO: 21), a peptide targeting hepatocellular carcinoma cells and having the amino acid sequence FQHPSFI (SEQ ID NO: 22), a peptide targeting integrin receptor and having the amino acid sequence RGD (SEQ ID NO: 23), a peptide targeting tumor neovasculature and having the amino acid sequence NGR (SEQ ID NO: 24), a peptide targeting endothelial VCAM-1 expressing cells and having the amino acid sequence VHSPNKK (SEQ ID NO: 25), a peptide targeting adenocarcinoma cells and having the amino acid sequence RRPYIL (SEQ ID NO: 26), a peptide targeting various carcinoma and having the amino acid sequence EDYELMDLLAYL (SEQ ID NO: 27), a peptide targeting breast carcinoma and having the amino acid sequence LTVSPWY (SEQ ID NO: 28), and a peptide targeting tumor neovasculature and having the amino acid sequence ATWLPPR (SEQ ID NO: 29).

22. The amphiphilic fusion protein of claim 20, wherein the -T- is an antimicrobial peptide selected from the group consisting of a dermcidin, an apidaecin, a bactenecin, and a pyrrhocoricin.

23. The amphiphilic fusion protein of claim 22, wherein:
(a) the dermcidin is a dermcidin variant selected from the group consisting of
DCD-1L comprising the amino acid sequence
(SEQ ID NO: 30)
SSLLEKGLDGAKKAVGGLGKLGKDAVEDLESVGKGAVHDVK
DVLDSVL,
DCD-1 comprising the amino acid sequence
(SEQ ID NO: 31)
SSLLEKGLDGAKKAVGGLGKLGKDAVEDLESVGKGAVHDVK
DVLDSV,
and
SSL25 comprising the amino acid sequence
(SEQ ID NO: 32)
SSLLEKGLDGAKKAVGGLGKLGKDA;
(b) the apidaecin comprises the amino acid sequence
(SEQ ID NO: 33)
GNNRP(V/I)YIPQPRPPHPR(L/I);
(c) the bactenecin is bactenecin 5 (Bac 5) or bactenecin 7 (Bac 7); or
(d) the pyrrhocoricin comprises the amino acid sequence
(SEQ ID NO: 34)
VDKGSYLPRPTPPRPIYNRN.

24-26. (canceled)

27. The amphiphilic fusion protein of claim 1, wherein:
(a) the —H₁—H₂ comprises an amino acid sequence selected from the group consisting of SEQ ID NO: 39, SEQ ID NO: 42, SEQ ID NO: 56, and SEQ ID NO: 57; and wherein:
(i) the amphiphilic fusion protein has the formula of S—X—H₁—H₂—, wherein S— is a solubilizing moiety, —X— is a peptide sequence comprising a proteolytic cleavage site, —H₁— is a hydrophilic peptide, and —H₂ is a hydrophobic peptide; or
(ii) the amphiphilic fusion protein has the formula of I—X—H₁—H₂, wherein I— is an insolubilizing moiety, —X— is a peptide sequence comprising a chemical cleavage site, —H₁— is a hydrophilic peptide, and —H₂ is a hydrophobic peptide.

28-29. (canceled)

30. An expression vector comprising a chimeric nucleic acid sequence encoding the amphiphilic fusion protein of claim 1.

31. A recombinant host cell engineered to express the amphiphilic fusion protein of claim 1, wherein the host cell is a eukaryotic, prokaryotic, archaea, mammalian, yeast, bacteria, cyanobacteria, insect, or plant cell.

32. The recombinant host cell of claim 31, wherein the bacteria cell is E. coli.

33. A method of producing an amphiphilic fusion protein that spontaneously self-assembles to form a stable micelle, the method comprising:
(a) introducing into a host cell an expression vector comprising a chimeric nucleic acid construct comprising, in the 5′ to 3′ direction, a promoter suitable for directing expression in a host cell operably linked to a nucleic acid sequence encoding an amphiphilic fusion protein having Formula (I): S/I—X—H₁—H₂, wherein S— is a solubilizing moiety, I— is an insolubilizing moiety, —X— is a peptide sequence comprising a proteolytic or chemical cleavage site, —H₁— is a hydrophilic peptide, and —H₂ is a hydrophobic peptide;
(b) growing the host cell under conditions that allow for expression of the chimeric nucleic acid to produce the amphiphilic fusion protein;
(c) purifying the amphiphilic fusion protein; and
(d) contacting the amphiphilic fusion protein with a protease or a reagent to induce chemical cleavage to provide an amphiphilic fusion protein having Formula (II): H₁—H₂.

34. The method of claim 33, wherein the chimeric nucleic acid construct of part (a) encodes an amphiphilic fusion protein further comprising a cell targeting peptide (-T-) between the —X— and the —H₁—, such that the amphiphilic fusion protein has Formula (III): S/I-X-T-H₁—H₂, and such that after part (d) the amphiphilic fusion protein has Formula (IV): T-H₁—H₂.

35-59. (canceled)

60. A micelle comprising an amphiphilic fusion protein comprising:
(i) a hydrophilic peptide (H₁); and
(ii) a hydrophobic peptide (H₂);
wherein the H₁ comprises an intrinsically disordered peptide (IDP) sequence selected from the group consisting of one or more polypeptide sequences from a human neurofilament protein, a San1 protein, an Hsp-33 protein, an E1A protein, a PhD protein, a Sic1 protein, a WASP protein, a p27 protein, a CREB protein, a PUP protein, and a LEA protein.

61-68. (canceled)

69. The micelle of claim 60, wherein the amphiphilic fusion protein further comprises a cell targeting peptide (T) covalently linked to the N-terminus of the H₁; and T is selected from the group consisting of a chitin binding domain (CBD), a cancer cell-targeting peptide, and an antimicrobial peptide.

70-76. (canceled)

77. The micelle of claim 60, wherein any one or more of the following:
(a) the critical micelle concentration (CMC) of the amphiphilic fusion protein in water is from about 10 μM to about 20 μM at a physiological pH of about 7.4;
(b) the micelle has a diameter from about 20 nm to about 40 nm;
(c) the micelle is stable at a pH from about 2.0 to about 10.0;
(d) the micelle is stable at a temperature from about 25° C. to about 70° C.
(e) the micelle further comprises a fluorescent dye, and the fluorescent dye is covalently attached to the hydrophilic peptide (H₁) or the hydrophobic peptide (H₂);
(f) the micelle has a core-shell structure having a shell diameter from about 40 nm to about 75 nm, a core diameter from about 25 nm to about 45 nm and a shell thickness from about 5 nm to about 20 nm;
(g) the micelle further comprises a hydrophobic cargo selected from a drug, a fungicide, a protein, a nucleic acid, a hormone, a receptor, a diagnostic agent, an imaging agent, a metal complex, a silicone oil, a triglyceride, or a combination thereof; or
(h) the amphiphilic fusion protein comprising H₁ and H₂ comprises an amino acid sequence selected from the group consisting of SEQ ID NO: 39, SEQ ID NO: 42, SEQ ID NO: 56, and SEQ ID NO: 57.

78-92. (canceled)

93. A pharmaceutical composition comprising a micelle of claim 60 and a hydrophobic cargo, wherein the hydrophobic cargo is a therapeutically active agent.

94. A method for treating a disease or disorder in a subject in need thereof comprising administering the pharmaceutical composition of claim 93 to the subject.

95. A composition suitable for use in drug delivery, cosmetics, paints and coatings, crop protection, nanoparticle synthesis and catalysis, home and personal care, and cleaning, comprising the micelle of claim 60.
